SEQUENCE LISTING (41 sequences)

SEQ ID NO: 1 - protein, 10 aa, synthetic construct
  source 1..10: note = Antibody 25A3 VH CDR1 (Exemplary)
GYTFDVYGIS

SEQ ID NO: 2 - protein, 17 aa, synthetic construct
  source 1..17: note = Antibody 25A3 VH CDR2 (Exemplary)
WIAPYSGNTNYAQKLQG

SEQ ID NO: 3 - protein, 14 aa, synthetic construct
  source 1..14: note = Antibody 25A3 VH CDR3 (Exemplary)
DAGTYSPFGYGMDV

SEQ ID NO: 4 - protein, 11 aa, synthetic construct
  source 1..11: note = Antibody 25A3 VL CDR1 (Exemplary)
QASQSINNWLA

SEQ ID NO: 5 - protein, 7 aa, synthetic construct
  source 1..7: note = Antibody 25A3 VL CDR2 (Exemplary)
KAYNLES

SEQ ID NO: 6 - protein, 10 aa, synthetic construct
  source 1..10: note = Antibody 25A3 VL CDR3 (Exemplary)
QLFQSLPPFT

SEQ ID NO: 7 - protein, 5 aa, synthetic construct
  source 1..5: note = Antibody 25A3 VH CDR1 (Kabat)
VYGIS

SEQ ID NO: 8 - protein, 17 aa, synthetic construct
  source 1..17: note = Antibody 25A3 VH CDR2 (Kabat)
WIAPYSGNTNYAQKLQG

SEQ ID NO: 9 - protein, 14 aa, synthetic construct
  source 1..14: note = Antibody 25A3 VH CDR3 (Kabat)
DAGTYSPFGYGMDV

SEQ ID NO: 10 - protein, 11 aa, synthetic construct
  source 1..11: note = Antibody 25A3 VL CDR1 (Kabat)
QASQSINNWLA

SEQ ID NO: 11 - protein, 7 aa, synthetic construct
  source 1..7: note = Antibody 25A3 VL CDR2 (Kabat)
KAYNLES

SEQ ID NO: 12 - protein, 10 aa, synthetic construct
  source 1..10: note = Antibody 25A3 VL CDR3 (Kabat)
QLFQSLPPFT

SEQ ID NO: 13 - protein, 7 aa, synthetic construct
  source 1..7: note = Antibody 25A3 VH CDR1 (Chothia)
GYTFDVY

SEQ ID NO: 14 - protein, 4 aa, synthetic construct
  source 1..4: note = Antibody 25A3 VH CDR2 (Chothia)
PYSG

SEQ ID NO: 15 - protein, 12 aa, synthetic construct
  source 1..12: note = Antibody 25A3 VH CDR3 (Chothia)
AGTYSPFGYGMD

SEQ ID NO: 16 - protein, 7 aa, synthetic construct
  source 1..7: note = Antibody 25A3 VL CDR1 (Chothia)
SQSINNW

SEQ ID NO: 17 - skipped

SEQ ID NO: 18 - protein, 7 aa, synthetic construct
  source 1..7: note = Antibody 25A3 VL CDR3 (Chothia)
FQSLPPF

SEQ ID NO: 19 - protein, 10 aa, synthetic construct
  source 1..10: note = Antibody 25A3 VH CDR1 (AbM)
GYTFDVYGIS

SEQ ID NO: 20 - protein, 10 aa, synthetic construct
  source 1..10: note = Antibody 25A3 VH CDR2 (AbM)
WIAPYSGNTN

SEQ ID NO: 21 - protein, 14 aa, synthetic construct
  source 1..14: note = Antibody 25A3 VH CDR3 (AbM)
DAGTYSPFGYGMDV

SEQ ID NO: 22 - protein, 11 aa, synthetic construct
  source 1..11: note = Antibody 25A3 VL CDR1 (AbM)
QASQSINNWLA

SEQ ID NO: 23 - protein, 7 aa, synthetic construct
  source 1..7: note = Antibody 25A3 VL CDR2 (AbM)
KAYNLES

SEQ ID NO: 24 - protein, 10 aa, synthetic construct
  source 1..10: note = Antibody 25A3 VL CDR3 (AbM)
QLFQSLPPFT

SEQ ID NO: 25 - protein, 6 aa, synthetic construct
  source 1..6: note = Antibody 25A3 VH CDR1 (Contact)
DVYGIS

SEQ ID NO: 26 - protein, 13 aa, synthetic construct
  source 1..13: note = Antibody 25A3 VH CDR2 (Contact)
WMGWIAPYSGNTN

SEQ ID NO: 27 - protein, 15 aa, synthetic construct
  source 1..15: note = Antibody 25A3 VH CDR3 (Contact)
ARDAGTYSPFGYGMD

SEQ ID NO: 28 - protein, 7 aa, synthetic construct
  source 1..7: note = Antibody 25A3 VL CDR1 (Contact)
NNWLAWY

SEQ ID NO: 29 - protein, 10 aa, synthetic construct
  source 1..10: note = Antibody 25A3 VL CDR2 (Contact)
LLIYKAYNLE

SEQ ID NO: 30 - protein, 9 aa, synthetic construct
  source 1..9: note = Antibody 25A3 VL CDR3 (Contact)
QLFQSLPPF

SEQ ID NO: 31 - protein, 8 aa, synthetic construct
  source 1..8: note = Antibody 25A3 VH CDR1 (IMGT)
GYTFDVYG

SEQ ID NO: 32 - protein, 8 aa, synthetic construct
  source 1..8: note = Antibody 25A3 VH CDR2 (IMGT)
IAPYSGNT

SEQ ID NO: 33 - protein, 16 aa, synthetic construct
  source 1..16: note = Antibody 25A3 VH CDR3 (IMGT)
ARDAGTYSPFGTGMDV

SEQ ID NO: 34 - protein, 6 aa, synthetic construct
  source 1..6: note = Antibody 25A3 VL CDR1 (IMGT)
QSINNW

SEQ ID NO: 35 - skipped

SEQ ID NO: 36 - protein, 10 aa, synthetic construct
  source 1..10: note = Antibody 25A3 VL CDR3 (IMGT)
QLFQSLPPFT

SEQ ID NO: 37 - protein, 123 aa, synthetic construct
  source 1..123: note = Antibody 25A3 VH Sequence
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSS

SEQ ID NO: 38 - protein, 108 aa, synthetic construct
  source 1..108: note = Antibody 25A3 VL Sequence
DIQMTQSPSTLSASVGDRVTITCQASQSINNWLAWYQQKPGKAPKLLIYKAYNLESGVPSRFSGSGSGTEFTLTISSLQPDDFATYYCQLFQSLPPFTFGGGTKVEIK

SEQ ID NO: 39 - protein, 219 aa, Homo sapiens
  source 1..219: note = Extracellular domain
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 40 - protein, 452 aa, synthetic construct
  source 1..452: note = Heavy chain sequence
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 215 aa, synthetic construct
  source 1..215: note = Light chain sequence
DIQMTQSPSTLSASVGDRVTITCQASQSINNWLAWYQQKPGKAPKLLIYKAYNLESGVPSRFSGSGSGTEFTLTISSLQPDDFATYYCQLFQSLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0099604 A1
(43) Pub. Date: Mar. 27, 2025

(54) PHARMACEUTICAL COMPOSITIONS COMPRISING ANTI-TISSUE FACTOR ANTIBODY-DRUG CONJUGATES

(71) Applicant: EXELIXIS, INC., Alameda, CA (US)

(72) Inventors: Mark REYNOLDS, Millbrae, CA (US); Hui LI, Clyde Hill, WA (US); Rajiv MAHAJAN, Boyds, MD (US)

(73) Assignee: EXELIXIS, INC., Alameda, CA (US)

(21) Appl. No.: 18/730,679

(22) PCT Filed: Jan. 19, 2023
(86) PCT No.: PCT/US2023/060921

§ 371 Date: Jul. 19, 2024

Related U.S. Application Data
(60) Provisional application No. 63/301,994, filed on Jan. 21, 2022.

(51) Int. Cl. A61K 47/68; A61K 47/22; A61K 47/26

(52) U.S. Cl. CPC A61K 47/6849; A61K 47/22; A61K 47/26; A61K 47/68031; A61K 47/6889

(57) ABSTRACT

Provided herein are pharmaceutical compositions comprising anti-tissue factor antibody-drug conjugates (ADCs) and pharmaceutically acceptable excipients.

### CROSS-REFERENCE TO RELATED APPLICATIONS

This application claims the benefit of U.S. Provisional Patent Application No. 63/301,994, filed Jan. 21, 2022, which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14529-102-228_SEQ_LISTING.xml”, was created on Jan. 18, 2023 and is 43,585 bytes in size.

### FIELD

The present disclosure relates to pharmaceutical compositions comprising anti-tissue factor antibody-drug conjugates (ADCs).

### BACKGROUND

Blood coagulation involves a complex set of processes that result in blood clotting. Tissue factor (TF) plays an important role in these coagulation processes. TF is a cell surface receptor for the serine protease factor VIIa (FVIIa). The TF/FVIIa complex catalyzes conversion of the inactive protease factor X (FX) into the active protease factor Xa (FXa). FXa and its co-factor FVa form the prothrombinase complex, which generates thrombin from prothrombin. Thrombin converts soluble fibrinogen into insoluble strands of fibrin and catalyzes many other coagulation-related processes.

TF is over-expressed on multiple types of solid tumors. In cancer, TF/FVIIa signaling can support angiogenesis, tumor progression, and metastasis.

There is a need in the art for stable formulations comprising anti-tissue factor antibody-drug conjugates (ADCs) useful for treating a disease or disorder such as solid tumors and cancer. The present disclosure addresses such a need.

### SUMMARY

Provided herein are pharmaceutical compositions comprising anti-tissue factor antibody-drug conjugates.

In some embodiments, provided herein is a pharmaceutical composition comprising:
(i) an antibody-drug conjugate of the following Formula (I):

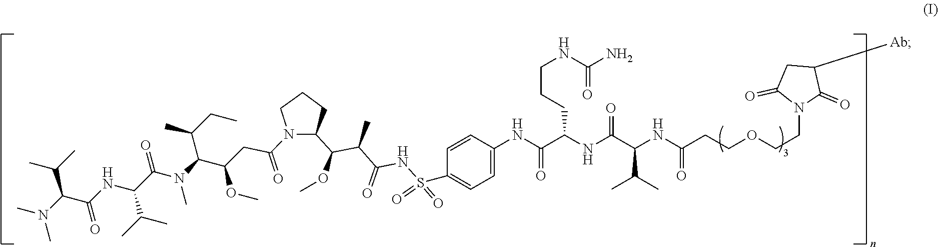

or a pharmaceutically acceptable salt thereof,
wherein:
   Ab is a tissue factor (TF) antibody, wherein the Ab comprises a VH-CDR1, a VH-CDR2, a VH-CDR3, a VL-CDR1, a VL-CDR2, and a VL-CDR3, wherein the VH-CDR1, VH-CDR2, VH-CDR3, VL-CDR1, VL-CDR2, and VL-CDR3 are from the antibody designated 25A3;
   n is an integer greater than or equal to 1; and
   the succinimidyl group is attached to the Ab through a covalent bond;
(ii) a buffer;
(iii) a tonicifier; and
(iv) a surfactant.

In some embodiments, the succinimidyl group is attached to the Ab via the cysteine residues of the Ab.

In some embodiments, the buffer is histidine, the tonicifier is sucrose, and the surfactant is polysorbate 80. In some embodiments, the pharmaceutical compositions provided herein comprise the antibody-drug conjugate provided herein, histidine, hydrochloric acid, sucrose, and polysorbate 80.

In some embodiments, provided herein is a pharmaceutical composition comprising 5-20 mg/mL of the antibody-drug conjugate provided herein, 10-50 mM histidine, 5-10% (w/v) sucrose, and 0.01-0.05% (w/v) polysorbate 80, and wherein the pharmaceutical composition has a pH value of between 5 to 6.

In some embodiments, provided herein is a pharmaceutical composition comprising 10 mg/mL of the antibody-drug conjugate provided herein, 20 mM histidine, 8% (w/v) sucrose, and 0.02% (w/v) polysorbate 80, and wherein the pharmaceutical composition has a pH value of about 5.5.

In another aspect, provided herein is a pharmaceutical composition provided herein for use in treating a disease or disorder in a subject. In some embodiments, the pharmaceutical composition is lyophilized. In some embodiments, the pharmaceutical composition is stored in a glass vial or a polycarbonate bottle.

In another aspect, provided herein is a method of treating a disease or disorder in a subject comprising administering the pharmaceutical composition provided herein to the subject. In some embodiments, the disease or disorder is cancer. In some embodiments, the cancer is selected from the group consisting of: non-small cell lung cancer (NSCLC), urothelial cancer, ovarian cancer (e.g., epithelial), cervical cancer (e.g., with squamous cell or adenocarcinoma histology), head and neck cancer (e.g., with squamous cell histology), and pancreatic cancer. In some embodiments, the subject is a human subject.

### BRIEF DESCRIPTION OF THE DRAWINGS

FIGS. 1A-1C show changes in percent monomer, percent high molecular weight (HMW), and percent low molecular weight (LMW) species measured by SEC for formulations prepared in different buffers and stored for up to 2 weeks at 40° C./75% RH. FIG. 1A shows few changes in molecular size distribution in histidine buffer. FIG. 1B shows apparent changes in molecular size distribution in citrate buffer. FIG. 1C shows apparent changes in molecular size distribution in phosphate buffer. In each of these figures, the three bars for each tested formulation represent storage times of T0, 1 wk, and 2 wk, respectively, from left to right.

FIGS. 2A-2D show the statistical significance of percent monomer changes during storage at 40° C. for up to 2 weeks as a function of buffer type, concentration (mM), and pH. FIG. 2A shows the formulations prepared with citrate and histidine had fewer changes in the percent monomer than the formulation prepared with phosphate. FIG. 2B shows 30 mM concentration exhibited fewer changes in percent monomer than the 10 mM and 50 mM concentrations. FIG. 2C shows smaller differences in percent monomer occurred at pH 5 and pH 6. FIG. 2D shows that the formulations prepared with histidine exhibited fewer changes in percent monomer during storage at 40° C. for up to 2 weeks.

FIG. 3 shows the changes in percent main peak analyzed by iCE. The formulations were prepared with histidine and stored at −20° C. or −80° C. for up to 6 months. Under both temperatures, formulation 1 that was prepared with 50 mM histidine at pH 7 exhibited a consistent decrease in percent main peak. The other formulations prepared with histidine showed no large changes in percent main peak.

FIG. 4 shows that all formulations prepared with citrate exhibited a percent acidic species increase during storage at 2° C. to 8° C. for up to 2 months. The four bars for each tested formulation represent storage times of T0, 2 weeks, 1 month, and 2 months, respectively, from left to right.

FIG. 5 shows that all formulations prepared with phosphate exhibited a percent main peak decrease during storage at 2° C. to 8° C. for up to 2 months. The four bars for each tested formulation represent storage times of T0, 2 weeks, 1 month, and 2 months, respectively, from left to right.

FIG. 6 shows the statistical significance of percent main peak changes as a function of buffer concentration (mM) for formulations during storage at 2° C. to 8° C. for up to 2 months.

FIG. 7 shows the statistical significance of percent main peak changes as a function of pH for formulations during storage at 2° C. to 8° C. for up to 2 months.

FIG. 8 shows the statistical significance of percent main peak changes as a function of pH for formulations stored at 25° C. for up to 2 weeks.

FIG. 9 shows the DLS analysis of average particle size and percent polydispersity for formulations at T0 (top) and after storage at −80° C. or −20° C. for up to 6 months (bottom). No large changes were observed for the tested formulations, except for formulations 1 and 4.

### DEFINITIONS

Unless otherwise defined, terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art.

As used herein, the singular forms “a,” “an,” and “the” include the plural referents unless the context clearly indicates otherwise. The terms “include,” “such as,” and the like are intended to convey inclusion without limitation, unless otherwise specifically indicated.

The term “about” or “approximately” indicates and encompasses an indicated value and a range above and below that value. In certain embodiments, the term “about” or “approximately” indicates the designated value±10%, 5%, or +1%. In certain embodiments, where applicable, the term “about” or “approximately” indicates the designated value(s)±one standard deviation of that value(s).

The terms “Tissue Factor” and “TF” are used interchangeably herein to refer to TF, or any variants (e.g., splice variants and allelic variants), isoforms, and species homologs of TF that are naturally expressed by cells, or that are expressed by cells transfected with a TF gene. In some aspects, the TF protein is a TF protein naturally expressed by a primate (e.g., a monkey or a human), a rodent (e.g., a mouse or a rat), a dog, a camel, a cat, a cow, a goat, a horse, a pig or a sheep. TF is a cell surface receptor for the serine protease factor Vila. It is often times constitutively expressed by certain cells surrounding blood vessels and in some disease settings. In certain embodiments, the antibodies and antibody-drug conjugates (ADCs) described herein bind to the extracellular domain of human Tissue Factor (TF) (SEQ ID NO:39).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, PA. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, MD, incorporated by reference in its entirety.

A “Complementary Determining Region (CDR)” refers to one of three hypervariable regions (H1, H2 or H3) within the non-framework region of the immunoglobulin (Ig or antibody) V_{H} β-sheet framework, or one of three hypervariable regions (L1, L2 or L3) within the non-framework region of the antibody VL β-sheet framework. CDRs are variable region sequences interspersed within the framework region sequences. CDRs are well recognized in the art and have been defined by, for example, Kabat as the regions of most hypervariability within the antibody variable (V) domains. See Kabat et al., J Biol Chem, 1977, 252:6609-6616 and Kabat, Adv Protein Chem, 1978, 32:1-75, each of which is incorporated by reference in its entirety. CDRs have also been defined structurally by Chothia as those residues that are not part of the conserved β-sheet framework, and thus are able to adapt different conformations. See Chothia and Lesk, J Mol Biol, 1987, 196:901-917, incorporated by reference in its entirety. Both the Kabat and Chothia nomenclatures are well known in the art. AbM, Contact and IMGT also define CDRs. CDR positions within a canonical antibody variable domain have been determined by comparison of numerous structures. See Morea et al., Methods, 2000, 20:267-279 and Al-Lazikani et al., J Mol Biol, 1997, 273:927-48, each of which is incorporated by reference in its entirety. Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra). Such terminology is well known to those skilled in the art.

A number of hypervariable region delineations are in use and are included herein. The Kabat CDRs are based on sequence variability and are the most commonly used. See Kabat et al. (1992) Sequences of Proteins of Immunological Interest, DIANE Publishing: 2719, incorporated by reference in its entirety. Chothia refers instead to the location of the structural loops (Chothia and Lesk, supra). The AbM hypervariable regions represent a compromise between the Kabat CDRs and Chothia structural loops, and are used by Oxford Molecular's AbM antibody modeling software. The Contact hypervariable regions are based on an analysis of the available complex crystal structures. The residues from each of these hypervariable regions are noted in Table 2, provided herein.

More recently, a universal numbering system ImMunoGeneTics (IMGT) Information System™ has been developed and widely adopted. See Lefranc et al., Dev Comp Immunol, 2003, 27:55-77, incorporated by reference in its entirety. IMGT is an integrated information system specializing in immunoglobulins (IG), T cell receptors (TR) and major histocompatibility complex (MHC) of human and other vertebrates. The IMGT CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. Correspondence between the Kabat, Chothia and IMGT numbering is also well known in the art (Lefranc et al., supra). An Exemplary system, shown herein, combines Kabat and Chothia CDR definitions.

The light chain from any vertebrate species can be assigned to one of two types, called kappa (κ) and lambda (λ), based on the sequence of its constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The term “subject” refers to a mammalian subject. Exemplary subjects include humans, monkeys, dogs, cats, mice, rats, cows, horses, camels, goats, rabbits, pigs and sheep. In some embodiments, the subject is a human. In some embodiments, the subject has a disease or condition that can be treated with an antibody provided herein.

The term “therapeutically effective amount” or “effective amount” refers to an amount of an antibody or pharmaceutical composition provided herein that, when administered to a subject, is effective to treat a disease or disorder.

### DETAILED DESCRIPTION

### Pharmaceutical Compositions

Provided herein is a pharmaceutical composition comprising an antibody-drug conjugate (ADC) and a pharmaceutically acceptable excipient. Also, provided herein is a pharmaceutical composition comprising a population of ADCs and a pharmaceutically acceptable excipient. The ADC and the population of ADCs are described in more detail below. In some embodiments, the excipient provided herein can be a buffer; a tonicifier; a surfactant, or a combination thereof.

In some embodiments, the pharmaceutical compositions provided herein comprise the antibody-drug conjugate(s) provided herein; a buffer; a tonicifier; and a surfactant. In some embodiments, the pharmaceutical compositions provided herein further comprise an agent that adjusts the pH value of the pharmaceutical composition. In some embodiments, the buffer is histidine, the tonicifier is sucrose, the surfactant is polysorbate 80, and/or the agent that adjusts the pH value of the pharmaceutical composition is hydrochloric acid.

In some embodiments, the pharmaceutical compositions provided herein comprise the antibody-drug conjugate(s) provided herein, histidine, hydrochloric acid, sucrose, and polysorbate 80. In some embodiments, the pharmaceutical compositions provided herein consist essentially of the antibody-drug conjugate(s) provided herein, histidine, hydrochloric acid, sucrose, and polysorbate 80.

In some embodiments, provided herein is a pharmaceutical composition comprising 5-20 mg/mL of the antibody-drug conjugate provided herein, 10-50 mM histidine, 5-10% (w/v) sucrose, and 0.01-0.05% (w/v) polysorbate 80, and wherein the pharmaceutical composition has a pH value of between 5 to 6. In some embodiments, provided herein is a pharmaceutical composition comprising 5-20 mg/mL of the antibody-drug conjugate provided herein, 10-30 mM histidine, 5-10% (w/v) sucrose, and 0.01-0.05% (w/v) polysorbate 80, and wherein the pharmaceutical composition has a pH value of between 5 to 6.

In some embodiments, provided herein is a pharmaceutical composition comprising 10 mg/mL of the antibody-drug conjugate provided herein, 20 mM histidine, 8% (w/v) sucrose, and 0.02% (w/v) polysorbate 80, and wherein the pharmaceutical composition has a pH value of about 5.5. In some embodiments, provided herein is a pharmaceutical composition consisting essentially of 10 mg/mL of the antibody-drug conjugate provided herein, 20 mM histidine, 8% (w/v) sucrose, and 0.02% (w/v) polysorbate 80, and wherein the pharmaceutical composition has a pH value of about 5.5.

In some embodiments, the pharmaceutical composition is lyophilized. Lyophilized powders can be reconstituted for administration as solutions, emulsions and other mixtures. They may also be reconstituted and formulated as solids or gels. Lyophilized powder may be prepared by dissolving the ADC provided herein in a suitable solvent. The solvent may contain an excipient which improves the stability or other pharmacological component of the powder or reconstituted solution, prepared from the powder. Subsequent sterile filtration of the solution can be performed under standard conditions known to those of skill in the art to provide the desired formulation. Generally, the resulting solution will be apportioned into vials for lyophilization. Each vial will contain a single dosage (including but not limited to 10-1000 mg or 100-500 mg) or multiple dosages of the ADC. The lyophilized powder can be stored under appropriate conditions, such as at about 4° C. to room temperature. In some embodiments, reconstitution of this lyophilized powder with water or another suitable carrier for injection provides a formulation for use in parenteral administration. For reconstitution, about 1-50 mg, about 5-35 mg, or about 9-30 mg of lyophilized powder, is added per mL of sterile water or another suitable carrier. The precise amount can be adjusted and empirically determined.

As used herein, the term “lyophilized” refers to the composition having been freeze-dried under a vacuum. Lyophilization typically is accomplished by freezing a particular formulation such that the solutes are separated from the solvent(s). The solvent is usually then removed by sublimation (i.e., primary drying) and next by desorption (i.e., secondary drying). In some embodiments, a lyophilization cycle is composed of three steps: freezing, primary drying, and secondary drying. See, e.g., A. P. Mackenzie, Phil Trans R Soc London, Ser B, Biol 278:167 (1977). In a typical freezing step, the solution is cooled to initiate ice formation. Furthermore, this step induces the crystallization of the bulking agent. The ice sublimes in the primary drying stage, which is conducted by reducing chamber pressure below the vapor pressure of the ice, using a vacuum and introducing heat to promote sublimation. Finally, adsorbed or bound water is usually removed at the secondary drying stage under reduced chamber pressure and at an elevated shelf temperature. In some embodiments, the process produces a material known as a lyophilized cake. In various embodiments, the cake can be reconstituted with either sterile water or suitable diluent for administration as described herein.

In some embodiments, the pharmaceutical composition is stored in a glass vial. In other embodiments, the pharmaceutical composition is stored in a polycarbonate bottle.

### Anti-Tissue Factor Antibodies

The ADC in the present pharmaceutical compositions comprises an antibody that specifically binds to tissue factor (TF).

In certain embodiments, the antibody of the ADC specifically binds to the extracellular domain of human TF (SEQ ID NO:39).

In certain embodiments, the antibody of the ADC comprises a heavy chain sequence. An illustrative heavy chain sequence is provided in Table 1. The heavy chain sequence may be a heavy chain sequence from the antibody clone identified as 25A3.

In certain embodiments, the antibody of the ADC comprises a light chain sequence. An illustrative light chain sequence is provided in Table 1. The light chain sequence may be a light chain sequence from the antibody clone identified as 25A3.

TABLE 1
Antibody 25A3-CDR Sequences

| | | Exemplary* | Kabat | Chothia | AbM | Contact | IMGT |
|---|---|---|---|---|---|---|---|
| VH | VH | GYTFDVYGIS | VYGIS | GYTFDVY | GYTFDVYGIS | DVYGIS | GYTFDVYG |
| CDR | CDR1 | (SEQ ID NO: 1) | (SEQ ID | (SEQ ID | (SEQ ID NO: 19) | (SEQ ID | (SEQ ID |
| Seq. | | | NO: 7) | NO: 13) | | NO: 25) | NO: 31) |
| | VH | WIAPYSGNTNYA | WIAPYSGNTNYA | PYSG | WIAPYSGNTN | WMGWIAPYSGN | IAPYSGNT |
| | CDR2 | QKLQG | QKLQG | (SEQ ID | (SEQ ID NO: 20) | TN | (SEQ ID |
| | | (SEQ ID NO: 2) | (SEQ ID | NO: 14) | | (SEQ ID | NO: 32) |
| | | | NO: 8) | | | NO: 26) | |
| | VH | DAGTYSPFGYG | DAGTYSPFGYG | AGTYSPFGYGM | DAGTYSPFGYG | ARDAGTYSPFG | ARDAGTYSPFG |
| | CDR3 | MDV | MDV | D | MDV | YGMD | TGMDV |
| | | (SEQ ID NO: 3) | (SEQ ID | (SEQ ID | (SEQ ID NO: 21) | (SEQ ID | (SEQ ID |
| | | | NO: 9) | NO: 15) | | NO: 27) | NO: 33) |
| VL | VL | QASQSINNWLA | QASQSINNWLA | SQSINNW | QASQSINNWLA | NNWLAWY | QSINNW |
| CDR | CDR1 | (SEQ ID NO: 4) | (SEQ ID | (SEQ ID | (SEQ ID NO: 22) | (SEQ ID | (SEQ ID |
| Seq. | | | NO: 10) | NO: 16) | | NO: 28) | NO: 34) |
| | VL | KAYNLES | KAYNLES | KAY | KAYNLES | LLIYKAYNLE | KAY |
| | CDR2 | (SEQ ID NO: 5) | (SEQ ID | (SEQ ID | (SEQ ID NO: 23) | (SEQ ID | (SEQ ID |
| | | | NO: 11) | NO: 17) | | NO: 29) | NO: 35) |
| | VL | QLFQSLPPFT | QLFQSLPPFT | FQSLPPF | QLFQSLPPFT | QLFQSLPPF | QLFQSLPPFT |
| | CDR3 | (SEQ ID NO: 6) | (SEQ ID | (SEQ ID | (SEQ ID NO: 24) | (SEQ ID | (SEQ ID |
| | | | NO: 12) | NO: 18) | | NO: 30) | NO: 36) |

VH Sequence*:
QVQLVQSGAEVKKPGASVKVSCKASGYTFDVYGISWVRQAPGQGLEWMGWIAPYSGNTNYAQKLQGRVTMTTDTSTSTAYMELRSLRSD
DTAVYYCARDAGTYSPFGYGMDVWGQGTTVTVSS (SEQ ID NO: 37)
VL Sequence*:
DIQMTQSPSTLSASVGDRVTITCQASQSINNWLAWYQQKPGKAPKLLIYKAYNLESGVPSRFSGSGSGTEFTLTISSLQPDDFATYYCQLFQS
LPPFTFGGGTKVEIK (SEQ ID NO: 38)
*Exemplary CDR sequences encompass amino acids as determined by Kabat plus Chothia. The exemplary CDR sequences are bolded.

In certain embodiments, the antibody of the ADC comprises a V_{H} sequence of SEQ ID NO:37. In certain embodiments, the ADC administered in the methods of treating provided herein comprises an antibody comprising a V_{H} sequence having at least about 50%, 60%, 70%, 80%, 90%, 95%, or 99% identity to an illustrative V_{H} sequence of SEQ ID NO:37.

In certain embodiments, the antibody of the ADC comprises a V_{L} sequence of SEQ ID NO:38. In certain embodiments, an antibody of the ADC administered in the methods of treating provided herein comprises a V_{L} sequence having at least about 50%, 60%, 70%, 80%, 90%, 95%, or 99% identity to an illustrative V_{L} sequence of SEQ ID NO:38.

In certain embodiments, the antibody of the ADC comprises a V_{H} sequence of SEQ ID NO:37, and a V_{L} sequence of SEQ ID NO:38.

In certain embodiments, the antibody of the ADC comprises a V_{H} sequence of SEQ ID NO:37 and a V_{L} sequence of SEQ ID NO:38. In certain embodiments, the antibody of the ADC comprises a V_{H} sequence having at least about 50%, 60%, 70%, 80%, 90%, 95%, or 99% identity to an illustrative V_{H} sequence of SEQ ID NO:37, and a V_{L} sequence having at least about 50%, 60%, 70%, 80%, 90%, 95%, or 99% identity to an illustrative V_{L} sequence of SEQ ID NO:38.

In certain embodiments, the antibody of the ADC comprises a heavy chain sequence of SEQ ID NO:40 and a light chain sequence of SEQ ID NO:41.

In some embodiments, the antibody of the ADC contains substitutions (e.g., conservative substitutions), insertions, or deletions relative to the reference sequence, but the antibody retains the ability to bind to TF. In some embodiments, a total of 1 to 10 amino acids have been substituted, inserted and/or deleted in a reference amino acid sequence. In some embodiments, substitutions, insertions, or deletions occur in regions outside the CDRs (e.g., in the FRs). Antibodies generated by conservative amino acid substitutions are included in the present disclosure. In a conservative amino acid substitution, an amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties. Optionally, the antibody provided herein includes post-translational modifications of a reference sequence.

In certain embodiments, the ADC comprises an anti-tissue factor antibody that comprises a heavy chain CDR sequence from antibody clone 25A3. Antibody 25A3 CDR sequences as determined by the Exemplary, Kabat, Chothia, AbM, Contact, and IMGT numbering systems are shown in Table 2.

TABLE 2
Antibody 25A3 CDR sequences as determined by the Exemplary,
Kabat, Chothia, AbM, Contact, and IMGT numbering systems

| | Exemplary | | | | | |
|---|---|---|---|---|---|---|
| | (Kabat + | | | | | |
| | Chothia) | Kabat | Chothia | AbM | Contact | IMGT |
| VH CDR1 | 26-35 | 31-35 | 26-32 | 26-35 | 30-35 | 27-38 |
| VH CDR2 | 50-65 | 50-65 | 52a-55 | 50-58 | 47-58 | 56-65 |
| VH CDR3 | 95-102 | 95-102 | 96-101 | 95-102 | 93-101 | 105-117 |
| VL CDR1 | 24-34 | 24-34 | 26-32 | 24-34 | 30-36 | 27-38 |
| VL CDR2 | 50-56 | 50-56 | 50-52 | 50-56 | 46-55 | 56-65 |
| VL CDR3 | 89-97 | 89-97 | 91-96 | 89-97 | 89-96 | 105-117 |

In certain embodiments, the ADC comprises a CDR-H3 sequence that is 50%, 75%, 80%, 85%, 90%, 95%, or 100% identical to the CDR-H3 sequence from antibody clone 25A3. In certain embodiments, the ADC comprises a CDR-H2 sequence that is 50%, 75%, 80%, 85%, 90%, 95%, or 100% identical to the CDR-H2 sequence from antibody clone 25A3. In certain embodiments, the ADC comprises a CDR-H1 sequence that is 50%, 75%, 80%, 85%, 90%, 95%, or 100% identical to the CDR-H1 sequence from antibody clone 25A3. In certain embodiments, the ADC comprises two heavy chain CDRs that are 50%, 75%, 80%, 85%, 90%, 95%, or 100% identical to the corresponding two heavy chain CDRs from antibody clone 25A3. In certain embodiments, the ADC comprises three heavy chain CDRs that are 50%, 75%, 80%, 85%, 90%, 95%, or 100% identical to the three heavy chain CDRs from antibody clone 25A3.

In certain embodiments, the ADC comprises an anti-tissue factor antibody that comprises a light chain CDR from antibody clone 25A3. In certain embodiments, the ADC comprises a CDR-L3 sequence that is 50%, 75%, 80%, 85%, 90%, 95%, or 100% identical to the CDR-L3 sequence from antibody clone 25A3. In certain embodiments, the ADC comprises a CDR-L2 sequence that is 50%, 75%, 80%, 85%, 90%, 95%, or 100% identical to the CDR-L2 sequence from antibody clone 25A3. In certain embodiments, the ADC comprises a CDR-L1 sequence that is 50%, 75%, 80%, 85%, 90%, 95%, or 100% identical to the CDR-L1 sequence from antibody clone 25A3. In certain embodiments, the ADC comprises two light chain CDRs that are 50%, 75%, 80%, 85%, 90%, 95%, or 100% identical to the corresponding two light chain CDRs from antibody clone 25A3. In certain embodiments, the ADC comprises three light chain CDRs that are 50%, 75%, 80%, 85%, 90%, 95%, or 100% identical to the three light chain CDRs from antibody clone 25A3.

In certain embodiments, the ADC comprises a CHR—H3 sequence that is 50%, 75%, 80%, 85%, 90%, 95%, or 100% identical to the CDR-H3 sequence from antibody clone 25A3 and a CDR-L3 sequence that is 50%, 75%, 80%, 85%, 90%, 95%, or 100% identical to the CDR-L3 sequence from antibody clone 25A3. In certain embodiments, the ADC comprises six CDR sequences that are 50%, 75%, 80%, 85%, 90%, 95%, or 100% identical to the corresponding six CDRs from antibody clone 25A3.

### Linker-Toxins

The ADC provided herein comprises a cytotoxic agent, for example, an auristatin derivative.

In certain embodiments, the auristatin derivative (toxin) is a moiety derived from the following Compound 9:

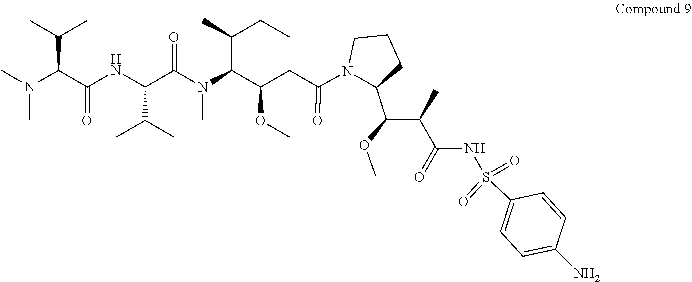 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C]

In certain embodiments, the ADC of the present disclosure comprises a TF antibody conjugated to an auristatin derivative (toxin) via a linker (L). In certain embodiments, the ADC comprises: (a) an antigen binding protein (Ab) which binds to the extracellular domain of human Tissue Factor (TF), wherein the Ab comprises a VH-CDR1, a VH-CDR2, a VH-CDR3, a VL-CDR1, a VL-CDR2, and a VL-CDR3, wherein the VH-CDR1, VH-CDR2, VH-CDR3, VL-CDR1, VL-CDR2, and VL-CDR3 are from the antibody designated 25A3; and (b) one or more linker-toxin moieties represented by Formula (III):

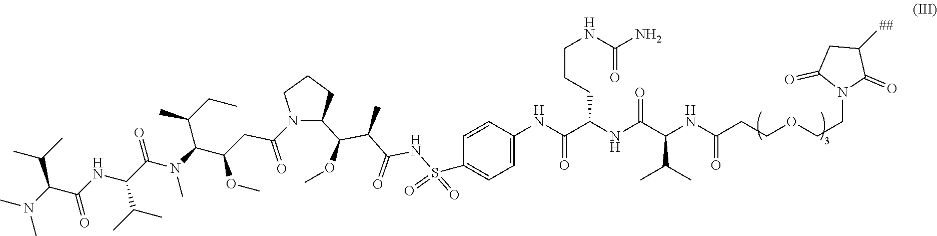

wherein ##represents the point of attachment of the linker-toxin moiety to the TF antibody and the linker-toxin moiety is attached to the TF antibody through a covalent bond.

### Antibody-Drug Conjugates

In some embodiments, the ADC provided herein is of the following Formula (I):

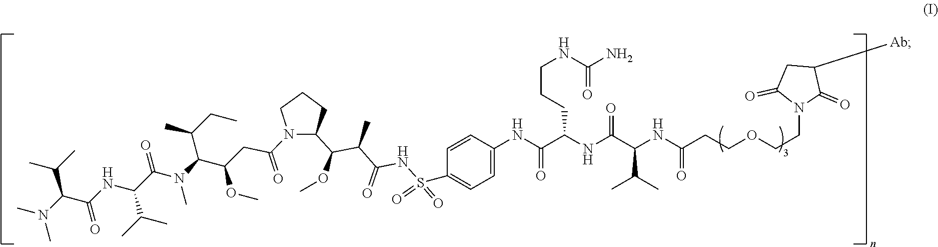

or a pharmaceutically acceptable salt thereof.

In certain embodiments, Ab is a tissue factor (TF) antibody, wherein the Ab comprises a VH-CDR1, a VH-CDR2, a VH-CDR3, a VL-CDR1, a VL-CDR2, and a VL-CDR3, wherein the VH-CDR1, VH-CDR2, VH-CDR3, VL-CDR1, VL-CDR2, and VL-CDR3 are from the antibody designated 25A3.

In certain embodiments, n is an integer greater than or equal to 1. In certain embodiments, the succinimidyl group is attached to the Ab through a covalent bond. In further embodiments, the succinimidyl group is attached to the Ab via cysteine residues of the Ab.

In certain embodiments, n is selected from the group consisting of 1, 2, 3, and 4. In certain embodiments, n is selected from the group consisting of 3 and 4.

In certain embodiments, the Ab comprises: a VH that is SEQ ID NO:37 and a VL sequence that is SEQ ID NO:38.

In certain embodiments, the Ab comprises:
a heavy chain sequence that is

(SEQ ID NO: 40)
QVQLVQSGAEVKKPGASVKVSCKASGYTFDVYGISWVRQAPGQGLEW
MGWIAPYSGNTNYAQKLQGRVTMTTDTSTSTAYMELRSLRSDDTAVYY
CARDAGTYSPFGYGMDVWGQGTTVTVSSASTKGPSVFPLAPSSKSTS
GGTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVM
HEALHNHYTQKSLSLSPG;
and
a light chain sequence that is
(SEQ ID NO: 41)
DIQMTQSPSTLSASVGDRVTITCQASQSINNWLAWYQQKPGKAPKLLIY
KAYNLESGVPSRFSGSGSGTEFTLTISSLQPDDFATYYCQLFQSLPPFT
FGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC.

### Populations of Antibody-Drug Conjugates

Also provided herein are pharmaceutical compositions comprising populations of antibody-drug conjugate (ADC) provided herein, wherein the ADC is of the following Formula (I):

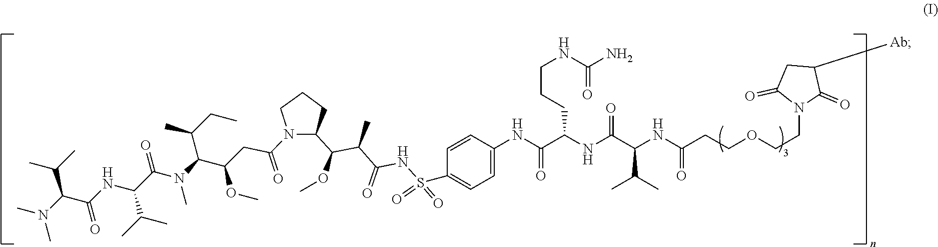

or a pharmaceutically acceptable salt thereof; wherein the population is a mixed population of antibody-drug conjugates in which n varies from 1 to 4. In certain embodiments, the average n of the population is about 3.8.

In certain embodiments, Ab is a tissue factor (TF) antibody, wherein the Ab comprises a VH-CDR1, a VH-CDR2, a VH-CDR3, a VL-CDR1, a VL-CDR2, and a VL-CDR3, wherein the VH-CDR1, VH-CDR2, VH-CDR3, VL-CDR1, VL-CDR2, and VL-CDR3 are from the antibody designated 25A3.

In certain embodiments, the Ab comprises: a VH that is SEQ ID NO:37 and a VL sequence that is SEQ ID NO:38.

In certain embodiments, the Ab comprises:

a heavy chain sequence that is
(SEQ ID NO: 40)
QVQLVQSGAEVKKPGASVKVSCKASGYTFDVYGISWVRQAPGQGLEW
MGWIAPYSGNTNYAQKLQGRVTMTTDTSTSTAYMELRSLRSDDTAVYY
CARDAGTYSPFGYGMDVWGQGTTVTVSSASTKGPSVFPLAPSSKSTS
GGTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVM
HEALHNHYTQKSLSLSPG;
and
a light chain sequence that is
(SEQ ID NO: 41)
DIQMTQSPSTLSASVGDRVTITCQASQSINNWLAWYQQKPGKAPKLLIY
KAYNLESGVPSRFSGSGSGTEFTLTISSLQPDDFATYYCQLFQSLPPFT
FGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

### Drug-Antibody Ratios

The number of linker-toxin moieties conjugated to an antibody in an ADC is defined as the drug-antibody ratio or DAR. As is known in the art, the majority of conjugation methods yield an ADC composition that includes various DAR species, due to the mixed population of ADCs, with the reported DAR being the average of the individual DAR species (where n is 1, 2, 3, 4, etc.). Thus, when the ADCs described herein are defined as having a specific DAR, it is to be understood that the number provided represents the average of the individual DAR species in the ADC composition. In some embodiments, the DAR is measured by UV/vis spectroscopy, hydrophobic interaction chromatography (HIC), and/or reverse phase liquid chromatography separation with time-of-flight detection and mass characterization (RP-UPLC/Mass spectrometry). In some embodiments, distribution of drug-linked forms (for example, the fraction of DAR0, DAR1, DAR2, etc. species) may also be analyzed by various techniques known in the art, including MS (with or without an accompanying chromatographic separation step), hydrophobic interaction chromatography, reverse-phase HPLC or iso-electric focusing gel electrophoresis (IEF) (see, for example, Sun et al., Bioconj Chem., 28:1371-81 (2017); Wakankar et al., mAbs, 3:161-172 (2011)).

The drug-antibody ratio (DAR) of the ADC in the compositions described herein may be between about 1 to 4. In certain embodiments, the DAR is between about 2 to 4. In certain embodiments, the DAR is between about 3 to 4. In certain embodiments, the DAR is about 3.0, about 3.1, about 3.2, about 3.3, about 3.4, about 3.5, about 3.6, about 3.7, about 3.8, about 3.9, or about 4.0. In certain embodiments, the DAR is about 3.8.

### Therapeutic Uses

The pharmaceutical compositions provided herein may be administered to a human intravenously by infusion, for example, by continuous infusion over a period of time.

The pharmaceutical compositions provided herein may be useful for the treatment of a disease or disorder involving TF. In some embodiments, the disease or disorder is a disease or disorder that can benefit from treatment with an anti-TF antibody or ADC.

In some embodiments, the pharmaceutical compositions provided herein are provided for use as a medicament. In some embodiments, the pharmaceutical compositions provided herein are provided for use in the manufacture or preparation of a medicament. In some embodiments, the medicament is for the treatment of a disease or disorder that can benefit from an anti-TF antibody or ADC.

In some embodiments, provided herein is a method of treating a disease or disorder in a subject in need thereof by administering an effective amount of a pharmaceutical composition provided herein to the subject.

In some embodiments, the disease or disorder that can benefit from treatment with an anti-TF antibody or ADC is cancer. In some embodiments, the pharmaceutical compositions provided herein are provided for use as a medicament for the treatment of cancer. In some embodiments, the pharmaceutical compositions provided herein are provided for use in the manufacture or preparation of a medicament for the treatment of cancer. In some embodiments, provided herein is a method of treating cancer. In some embodiments, the cancer is selected from the group consisting of: non-small cell lung cancer (NSCLC), urothelial cancer, ovarian cancer (e.g., epithelial), cervical cancer (e.g., with squamous cell or adenocarcinoma histology), head and neck cancer (e.g., with squamous cell histology), and pancreatic cancer.

In some embodiments, provided herein is a method of delaying the onset of a cancer in a subject in need thereof by administering an effective amount of a pharmaceutical composition provided herein to the subject. In some embodiments, provided herein is a method for late intervention treatment of cancer in a subject in need thereof. For example, the pharmaceutical composition can reduce the size of a tumor (e.g., tumor volume) in a subject in need thereof or inhibit the growth of a tumor in a subject in need thereof.

In some embodiments, provided herein is a method of preventing the onset of a cancer in a subject in need thereof by administering an effective amount of a pharmaceutical composition provided herein to the subject.

In some embodiments, provided herein is a method of reducing the size of a tumor (e.g., tumor volume) in a subject in need thereof by administering an effective amount of a pharmaceutical composition provided herein to the subject. In some embodiments, a pharmaceutical composition provided herein reduces tumor size (e.g. tumor volume) by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%. In some embodiments, an ADC provided herein inhibits tumor growth by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

In some embodiments, provided herein is a method of reducing the number of metastases in a subject in need thereof by administering an effective amount of a pharmaceutical composition provided herein to the subject.

In some embodiments, provided herein is a method for extending the period of overall survival, median survival time, or progression-free survival in a subject in need thereof by administering an effective amount of a pharmaceutical composition provided herein to the subject.

In some embodiments, provided herein is a method for treating a subject who has become resistant to a standard of care therapeutic by administering an effective amount of a pharmaceutical composition provided herein to the subject.

In some embodiments, the disease or disorder is a disease or disorder involving neovascularization. In certain embodiments, the disease or disorder involving neovascularization is cancer. In some embodiments, the disease or disorder is a disease or disorder involving vascular inflammation.

In some embodiments, the pharmaceutical compositions provided herein are provided for use as a medicament for the treatment of a disease or disorder involving neovascularization. In some embodiments, the pharmaceutical compositions provided herein are provided for use in the manufacture or preparation of a medicament for the treatment of a disease or disorder involving neovascularization. In certain embodiments, the disease or disorder involving neovascularization is cancer. In some embodiments, the pharmaceutical compositions provided herein are provided for use as a medicament for the treatment of a disease or disorder involving vascular inflammation. In some embodiments, the pharmaceutical compositions provided herein are provided for use in the manufacture or preparation of a medicament for the treatment of a disease or disorder involving vascular inflammation.

In some embodiments, provided herein is a method of treating a disease or disorder involving neovascularization in a subject in need thereof by administering an effective amount of a pharmaceutical composition provided herein to the subject. In certain embodiments, the disease or disorder involving neovascularization is cancer. In some embodiments, provided herein is a method of treating a disease or disorder involving vascular inflammation in a subject in need thereof by administering an effective amount of a pharmaceutical composition provided herein to the subject.

In some embodiments, provided herein is a method of delaying the onset of a disease or disorder involving neovascularization in a subject in need thereof by administering an effective amount of a pharmaceutical composition provided herein to the subject.

In some embodiments, provided herein is a method of preventing the onset of a disease or disorder involving neovascularization in a subject in need thereof by administering an effective amount of a pharmaceutical composition provided herein to the subject.

In some embodiments, provided herein is a method of delaying the onset of a disease or disorder involving vascular inflammation in a subject in need thereof by administering an effective amount of a pharmaceutical composition provided herein to the subject.

In some embodiments, provided herein is a method of preventing the onset of a disease or disorder involving vascular inflammation in a subject in need thereof by administering an effective amount of a pharmaceutical composition provided herein to the subject.

It is understood that modifications which do not substantially affect the activity of the various embodiments described herein are also provided within the definition of the subject matter described herein. Accordingly, the following examples are intended to illustrate but not limit the present disclosure.

### EXAMPLES

The following Examples are intended as an illustration of the various embodiments disclosed herein. In certain embodiments, the compounds are prepared by a variety of synthetic routes. These examples are not intended, nor are they to be construed, as limiting the scope of the invention. All publications, patents, and patent applications cited herein are hereby incorporated by reference in their entireties for all purposes.

### Example 1: Synthesis of Linker-Toxin A

The following example describes the preparation of an exemplary linker-toxin (Linker-Toxin A, also referred to as LT-A) that comprises the auristatin derivative, Compound 9:

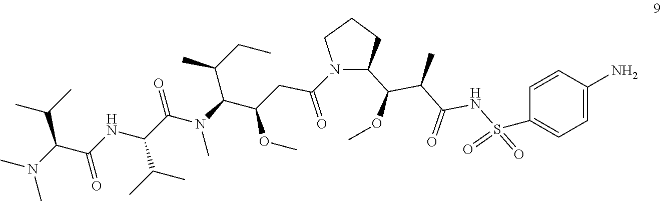 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C]

### 7.1 Ethyl (2R,3R)-3-methoxy-2-methyl-3-((S)-pyrrolidin-2-yl)propanoate (Compound 1)

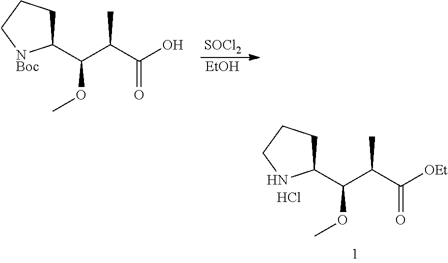

To a stirred solution of (2R,3R)-3-((S)-1-(tert-butoxycarbonyl)pyrrolidin-2-yl)-3-methoxy-2-methylpropanoic acid (Boc-Dap-OH, 4.31 g, 15.0 mmol) in absolute ethanol (27.0 mL) at 0° C. was added thionyl chloride (3.0 mL) in a dropwise fashion. The resulting solution was allowed to warm to room temperature and progress was monitored by HPLC-MS. After 18 h, no remaining starting material was detected and the solution was concentrated to dryness under reduced pressure. The resulting oil was suspended in toluene (10 mL) and concentrated under reduced pressure two times, then suspended in diethyl ether (5 mL) and concentrated under reduced pressure two times to afford the title compound as a white solid foam (3.78 g, quant yield %). MS m/z obs.=216.5 (M+1).

### 7.2 (3R,4S,5S)-4-((S)-2-(((benzyloxy)carbonyl)amino)-N,3-dimethylbutanamido)-3-methoxy-5-methylheptanoic acid (Compound 3)

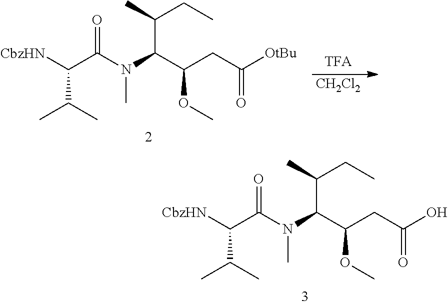

Compound 2 was prepared as described in International Patent Application Publication No. WO 2016/041082.

To a stirred solution of Compound 2 (6.965 g, 14.14 mmol) in dichloromethane (20 mL) was added trifluoroacetic acid (5.0 mL). The reaction was monitored for completion by HPLC-MS and after 40 h no starting material remained. The reaction was concentrated under reduced pressure, co-evaporated with toluene (2×10 mL) and dichloromethane (2×10 mL) to obtain a foamy white solid (6.2 g, quant yield with residual TFA). This material was dissolved in 200 mL of hot 1:3 EtOAc:hexanes and allowed to cool to room temperature. During cooling, a precipitate formed as well as some small crystals. 5 mL EtOAc was added and the suspension was heated once again to fully dissolve the precipitate. More crystals formed on cooling to room temperature and the flask was placed at −30° C. overnight. The following morning the mother liquor was decanted and the crystals rinsed with 2×50 mL hexanes and dried under high vacuum. Recovered 5.67 g of the title compound as a crystalline product. MS m/z obs.=405.7 (M+1).

### 7.3 Ethyl (2R,3R)-3-((S)-1-((3R,4S,5S)-4-((S)-2-(((benzyloxy)carbonyl)amino)-N,3-dimethylbutanamido)-3-methoxy-5-methylheptanoyl)pyrrolidin-2-yl)-3-methoxy-2-methylpropanoate (Compound 4)

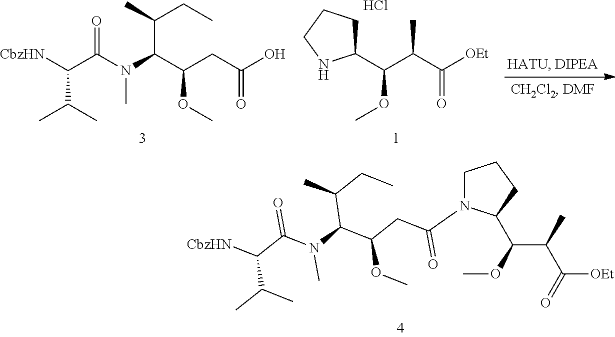

To a stirred solution of Compound 3 (6.711 g, 15.37 mmol, 1.025 equiv) in a mixture of dichloromethane (5.0 mL) and N,N-dimethylformamide (5.0 mL) at room temperature was added HATU (5.732 g, 15.07 mmol, 1.005 equiv) and N,N-diisopropylethylamine (7.84 mL, 3 equiv). After stirring for 30 minutes at room temperature, a solution of Compound 1 (3.776 g, 15.00 mmol, 1.0 equiv) in a mixture of dichloromethane (1.0 mL) and N,N-dimethylformamide (1.0 mL) was added dropwise and rinsed in residual Compound 1 with an additional 3 mL of 1:1 dichloromethane:N,N-dimethylformamide. The reaction was monitored by HPLC-MS and no remaining Compound 1 was observed after 15 minutes. The reaction was concentrated under reduced pressure, diluted with ethyl acetate (˜125 mL) and the organic phase was extracted with 1 M HCl (2×50 mL), 1×dH₂O (1×50 mL), saturated NaHCO₃ (3×50 mL), brine (25 mL). Acidic and basic aqueous layers were both washed with 25 mL EtOAc. All organics were then pooled and dried over MgSO₄, filtered and concentrated to give a red oil. The residue was dissolved in a minimal amount of dichloromethane (˜10 mL), loaded on to a Biotage© SNAP Ultra 360 g silica gel column (Isolera™ Flash System; Biotage AB, Sweden) for purification (20-100% EtOAc in hexanes over 10 column volumes). Fractions containing pure product were pooled to recover 7.9 g of foamy white solid. Impure fractions were subjected to a second purification on a Biotage® SNAP Ultra 100 g silica gel column and pooled with pure product to recover the title compound as a white foam solid (8.390 g, 88.3%). MS m/z obs.=634.7 (M+1).

### 7.4 (2R,3R)-3-((S)-1-((3R,4S,5S)-4-((S)-2-(((benzyloxy)carbonyl)amino)-N,3-dimethyl butanamido)-3-methoxy-5-methylheptanoyl)pyrrolidin-2-yl)-3-methoxy-2-methylpropanoic acid (Compound 5)

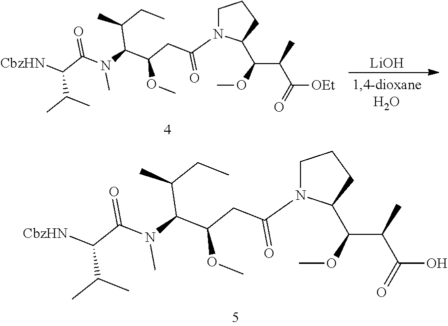 [SMILES: CCOC(=O)[C@H](C)[C@@H](OC)[C@@H]1CCCN1C(=O)C[C@@H](OC)[C@H]([C@@H](C)CC)N(C)C(=O)[C@@H](NC(=O)OCc1ccccc1)C(C)C ; "LiOH" ; CC[C@H](C)[C@@H]([C@@H](CC(=O)N1CCC[C@H]1[C@H](OC)[C@@H](C)C(=O)O)OC)N(C)C(=O)[C@@H](NC(=O)OCc1ccccc1)C(C)C]

To a stirred solution of Compound 4 (8.390 g, 13.24 mmol) in 1,4-dioxane (158 mL) was added dH₂O (39.7 ml) and lithium hydroxide monohydrate (1 M in H₂O, 39.7 mL, 3 equiv). The reaction was stirred at 4° C. and monitored by HPLC-MS for consumption of starting material, which took 3 days until only trace Compound 4 remained. During the course of the reaction, a new product, corresponding to loss of methanol (β-elimination, <2%) formed in small percentages in addition to the desired material. The reaction was acidified with the addition of 1 M aqueous HCl (50 mL) and concentrated under reduced pressure to remove the dioxane. The remaining reaction mixture was extracted with ethyl acetate (4×50 mL) and the organic phase was pooled, washed with brine (15 mL+2 mL 2 M HCl), dried over MgSO₄, filtered and concentrated under reduced pressure to yield a light colored oil. The oil was re-dissolved in diethyl ether (˜50 mL) and concentrated under reduced pressure (3×) to facilitate the removal of residual dioxane, affording the title product as a stiff oil (7.81 g 97% yield with some residual dioxane and Compound 4). MS m/z obs.=606.7 (M+1).

### 7.5 Benzyl ((S)-1-(((3R,4S,5S)-3-methoxy-1-((S)-2-((1R,2R)-1-methoxy-2-methyl-3-oxo-3-((4-(2,2,2-trifluoroacetamido)phenyl)sulfonamido)propyl)pyrrolidin-1-yl)-5-methyl-1-oxoheptan-4-yl)(methyl)amino)-3-methyl-1-oxobutan-2-yl)carbamate (Compound 7)

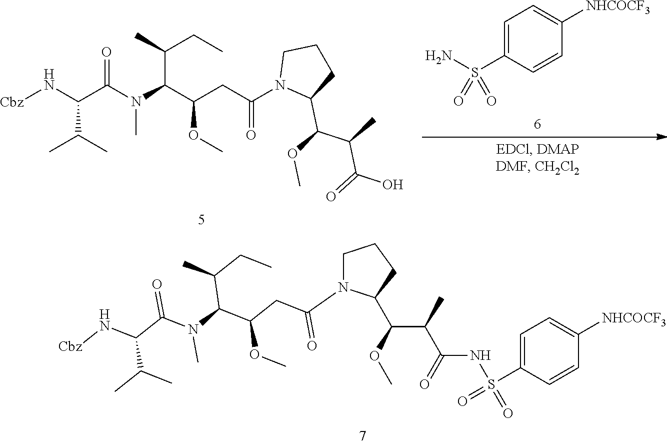

Compound 6 was prepared as described in International Patent Application Publication No. WO 2016/041082.

To a stirred solution of Compound 5 (7.12 g, 11.754 mmol) in dichloromethane (20 mL) was added 2,2,2-trifluoro-N-(4-sulfamoylphenyl)acetamide (Compound 6, 4.095 g, 1.3 equiv, dissolved in 3 mL DMF), N,N-dimethylpyridine (1.867 g, 1.3 equiv) and N,N-dimethylformamide (1.5 mL) to generate a light yellow suspension. Further addition of 5 mL of DMF did not clarify the solution. N-(3-Dimethylaminopropyl)-N′-ethylcarbodiimide hydrochloride (EDCl) (2.817 g, 1.25 equiv) was added in a single portion and the reaction was monitored by HPLC-MS. After 48 hr, reaction was no longer progressing and an additional 400 mg of EDCl was added. After 18 hr, no remaining starting material was observed and the reaction was concentrated under reduced pressure to give a yellow oil. The oil was dissolved in ethyl acetate (˜150 mL) and 1 M HCl (20 mL), and the organic phase was washed with cold 2 M HCl (2×10 mL), saturated NaHCO₃ (1×10 mL), brine (20 mL+5 mL 2 M HCl). Acidic and basic aqueous fractions were extracted with EtOAc (1×20 mL), all organic fractions were pooled, dried over MgSO₄ and concentrated under reduced pressure to yield an oily crude solid (13 g). The residue was dissolved in dichloromethane (˜10 mL), loaded on to a Biotage® SNAP Ultra 360 g silica gel column and purified under a 10-100% EtOAc (2% AcOH) in hexanes gradient over 12 column volumes with a 3-column volume plateau at 50% EtOAc. Fractions containing the pure product were pooled, concentrated under reduced pressure, dissolved and concentrated from toluene (2×10 mL) and diethyl ether (2×10 mL) to afford the desired product, 7.1 g of white foam solid. Impure fractions were subjected to repeat purification under shallower gradient conditions using a Biotage® SNAP Ultra 100 g silica gel column on an Isolera™ instrument. All pure fractions were pooled to recover pure product (the title compound) as a white foam solid (8.60 g, 86%). MS m/z obs.=856.7 (M+1).

### 7.6 (S)-2-amino-N-((3R,4S,5S)-3-methoxy-1-((S)-2-((1R,2R)-1-methoxy-2-methyl-3-oxo-3-((4-(2,2,2-trifluoroacetamido)phenyl)sulfonamido)propyl)pyrrolidin-1-yl)-5-methyl-1-oxoheptan-4-yl)-N,3-dimethylbutanamide (Compound 7a)

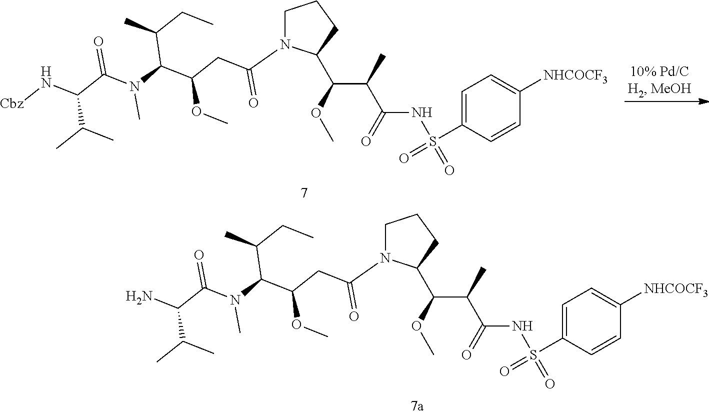

Compound 7 (3.71 g, 4.33 mmol) was dissolved in 10% N,N-dimethylformamide in ethyl acetate (30 mL) in a round bottom flask containing a magnetic stirrer and fitted with a 3-way gas line adapter. The vessel was twice evacuated under reduced pressure and charged with nitrogen gas. 10% palladium on carbon (0.461 g, 0.1 equiv) was added in a single portion, the 3-way adapter was fitted to the flask, a hydrogen balloon was fitted to the adapter and the vessel twice evacuated under reduced pressure and charged with hydrogen. The reaction was allowed to stir for 2 days, over which time the hydrogen balloon was occasionally recharged. After approximately 48 h, HPLC-MS analysis indicated that no starting material remained. The reaction was diluted with methanol (20 mL) and filtered through a plug of celite. The celite was washed with methanol (2×50 mL). All filtrates were pooled and concentrated under reduced pressure and the resulting oil dissolved and concentrated from dichloromethane. After drying under reduced pressure, the title compound was isolated as a colorless powder (3.10 g, 99%). MS m/z obs.=722.6 (M+1).

### 7.7 (S)-2-((S)-2-(dimethylamino)-3-methylbutanamido)-N-((3R,4S,5S)-3-methoxy-1-((S)-2-((1R,2R)-1-methoxy-2-methyl-3-oxo-3-((4-(2,2,2-trifluoroacetamido)phenyl)sulfonamido) propyl)pyrrolidin-1-yl)-5-methyl-1-oxoheptan-4-yl)-N,3-dimethylbutanamide (Compound 8)

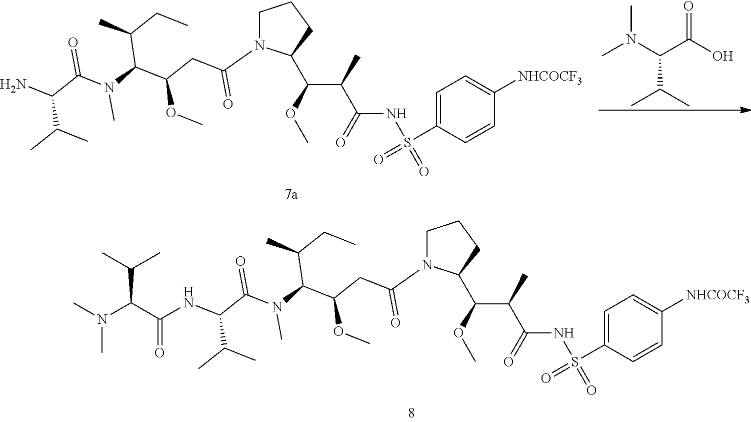

To a stirred solution of N,N-(L)-dimethylvaline (1.696 g, 9.35 mmol) in N,N-dimethylformamide (10 mL) was added HATU (3.216 g, 8.46 mmol) and diisopropylethylamine (3.10 mL, 17.8 mmol). A clear yellow solution resulted after 5 minutes. Stirring was continued for an additional 10 minutes, then Compound 7a (3.213 g, 4.45 mmol) was added in a single portion. After an additional 1 h of stirring, HPLC-MS indicated that trace amounts of Compound 7a remained and the reaction was for 16 h. The reaction was then concentrated under reduced pressure, diluted with ethyl acetate (120 mL) and 40 mL 1:1 NaHCO₃ (sat.): 5% LiCl and transferred to a separating funnel. The aqueous layer was removed and the organic phase was washed with LiCl (1×20 mL), NaHCO₃ (sat., 2×20 mL). Aqueous layers were pooled and extracted with EtOAc (3×50 mL). Organic layers were pooled and washed with brine (1×20 mL), dried over sodium sulfate, filtered and concentrated to give a DMF-laden oil which was concentrated via rotary evaporator to remove residual DMF, yielding 7 g of crude straw colored oil. The oil was dissolved in a minimal amount of 10% methanol in dichloromethane (˜11 mL) and loaded onto a Biotage© SNAP Ultra 360 g silica gel column for purification (2-20% MeOH in CH₂Cl₂ over 15 column volumes, product eluting around 10-13%). The fractions containing the desired product were pooled and concentrated under reduced pressure to afford the title compound as a colorless foam. Impure fractions were combined, evaporated and subjected to repeat purification on a Biotage® SNAP Ultra 100 g silica gel column on an Isolera™ instrument and combined with the pure product from the first column to yield a colorless foam solid (3.78 g). MS m/z obs.=850.6 (M+1).

### 7.8 (S)—N-((3R,4S,5R)-1-((S)-2-((1R,2R)-3-((4-aminophenyl)sulfonamido)-1-methoxy-2-methyl-3-oxopropyl)pyrrolidin-1-yl)-3-methoxy-5-methyl-1-oxoheptan-4-yl)-2-((S)-2-(dimethylamino)-3-methylbutanamido)-N,3-dimethylbutanamide (Compound 9)

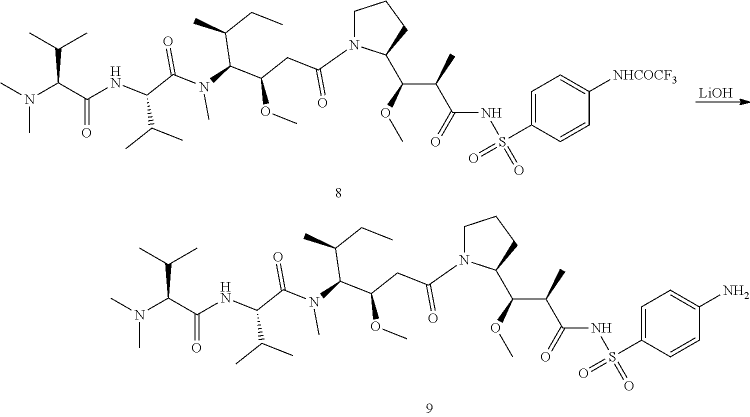

To a stirred solution of Compound 8 (0.980 g, 1.154 mmol) in 1,4-dioxanes (15 mL) was added water (3.5 mL) and 1 M lithium hydroxide monohydrate (3 equiv., 3.46 mL). The resulting light suspension was allowed to stir at 4° C. and was monitored by HPLC-MS for consumption of the starting material. When the conversion was complete (˜5 days), the reaction was neutralized with 3.46 mL of 1 M HCl and concentrated under reduced pressure to remove dioxane. The resulting aqueous phase was diluted with 60 mL EtOAc and 5 mL brine, then extracted with ethyl acetate (2×30 mL). The organic fractions were pooled, dried over Na₂SO₄, filtered and evaporated to yield the title compound as a tan solid (0.930 g). R_{f}=0.5 (8% MeOH in CH₂Cl₂). MS m/z obs.=753.7 (M+1).

### 7.9 2,3,5,6-tetrafluorophenyl 3-(2-(2-(2-(2,5-dioxo-2,5-dihydro-1H-pyrrol-1-yl)ethoxy)ethoxy)ethoxy)propanoate (Compound 15)

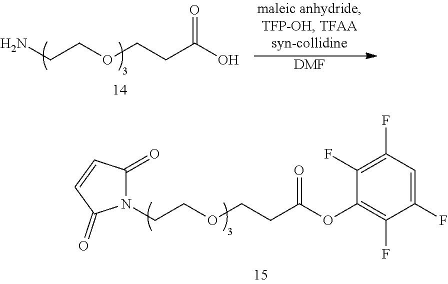

In a dried 50 mL conical flask, 3-(2-(2-(2-aminoethoxy)ethoxy)ethoxy)propanoic acid (Compound 14, 1.000 g, 4.52 mmol) and maleic anhydride (0.443 g, 4.52 mmol) were dissolved in anhydrous N,N-dimethylformamide (5 mL). The reaction was stirred at room temperature for 6 hr under N₂, then cooled to 0° C. and syn-collidine (1.263 mL, 2.1 eq) was added dropwise. In a separate dried 50 mL conical flask, tetrafluorophenol (3.002 g, 4 eq) was dissolved in anhydrous N,N-dimethylformamide (10 mL). The flask was cooled to 0° C. in an ice bath and trifluoroacetic anhydride (2.548 mL, 4 eq) was added dropwise. After stirring for 15 minutes, syn-collidine (2.407 mL, 4 eq) was added dropwise. The flask was allowed to stir for another 15 minutes, and then the contents were added to the first flask dropwise, via syringe. The reaction was allowed to warm to room temperature and stirring was continued under N₂. The reaction was monitored by HPLC-MS for the consumption of starting materials. After 6 days, the reaction was complete with the total consumption of Compound 14, leaving only Compound 15 and a small amount (˜5%) of the bis-TFP maleic amide intermediate. The reaction was transferred to a separating funnel, diluted with diethyl ether (75 ml) and washed with 5% LiCl (1×20 mL), 1 M HCl (2×20 mL), sat. NaHCO₃ (5×20 mL) and brine (1×20 mL). The organic layer was dried over Na₂SO₄, filtered and evaporated to give brown crude oil with residual DMF. Crude oil was dissolved in 8 mL of 1:1 DMF:H₂O+0.1% TFA, loaded onto a 60 g Biotage© SNAP Ultra C18 column (Biotage AB, Uppsala, Sweden) and purified under a linear 30-100% gradient of ACN/H₂O+0.1% TFA over 8 column volumes. Pure fractions were pooled and diluted with brine (20 mL), then extracted 3×50 mL Et₂O. Pooled organics were dried over MgSO₄, filtered and evaporated to recover the title compound as a light-yellow oil (1.34 g, 66% yield).

### 7.10 Tert-butyl ((S)-1-(((S)-1-((4-(N-((2R,3R)-3-((S)-1-((3R,4S,5S)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-2-methylpropanoyl)sulfamoyl)phenyl)amino)-1-oxo-5-ureidopentan-2-yl)amino)-3-methyl-1-oxobutan-2-yl)carbamate (Compound 12)

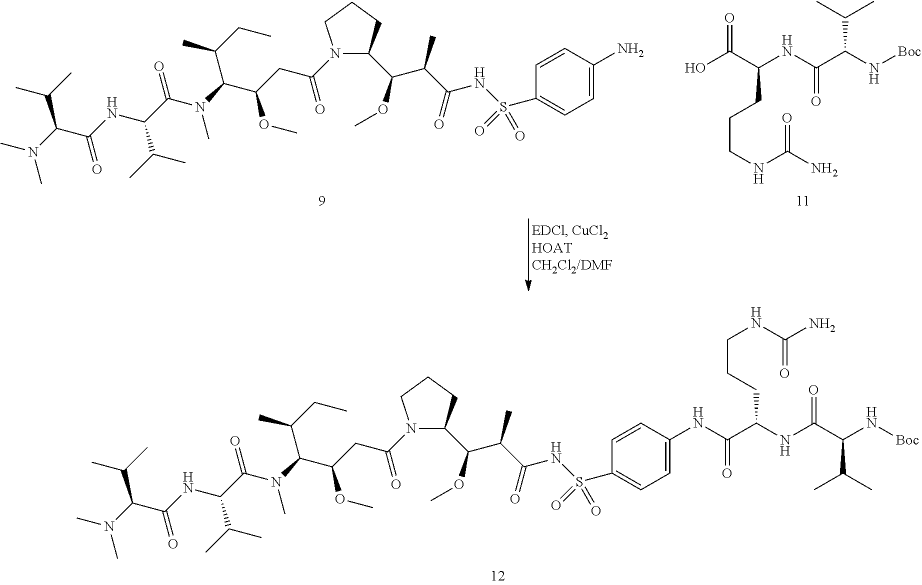 [SMILES: CC[C@H](C)[C@@H]([C@@H](CC(=O)N1CCC[C@H]1[C@H](OC)[C@@H](C)C(=O)NS(=O)(=O)c1ccc(N)cc1)OC)N(C)C(=O)[C@@H](NC(=O)[C@H](C(C)C)N(C)C)C(C)C ; CC(C)[C@H](NC(=O)OC(C)(C)C)C(=O)N[C@@H](CCCNC(N)=O)C(=O)O ; CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)OC(C)(C)C)C(C)C)cc1)OC)N(C)C(=O)[C@@H](NC(=O)[C@H](C(C)C)N(C)C)C(C)C]

Compound 11 was prepared as described in International Patent Application Publication No. WO 2016/041082.

To an empty 25 mL pear shaped flask, was added Compound 11 (1.342 g, 3.58 mmol, 3.0 equiv), 1-ethyl-3-(3-dimethylaminopropyl)carbodiimide hydrochloride (0.664 g, 3.46 mmol, 2.9 equiv) and 7-hydroxy-azabenzotriazole (HOAT) (0.472 g, 3.46 mmol, 2.9 equiv). These solids were dissolved in a mixture of N,N-dimethylformamide (0.5 mL) and dichloromethane (4.5 mL) with stirring at room temperature over 30 minutes. Separately, Compound 9 (0.900 g, 1.20 mmol) was dissolved in a mixture of N,N-dimethylformamide (0.2 mL) and dichloromethane (1.8 mL) and added to the pear shaped flask, rinsing with dichloromethane (1.0 mL). Stirring rate was increased to 1000 rpm, producing a vortex. Within 2 minutes of adding Compound 9, copper (II) chloride (0.514 g, 3.83 mmol, 3.2 equiv) was added in one portion directly into the center of the vortex through a narrow powder funnel. The initially light-yellow solution turned to a dark-brown suspension which changed over 10 minutes to a dark-green suspension. The reaction was monitored for completion by HPLC-MS and no change to reaction progress was observed between the samples taken at 30 minutes and 1 h (˜95% complete). The reaction was allowed to stir overnight at room temperature, then 2-(2-aminoethylamino)ethanol (0.483 mL, 4.781 mmol, 4 equiv), EtOAc (10 mL) and dH₂O (5 mL) were added to the stirred suspension, which underwent a color change to deep blue. The suspension was stirred vigorously for 4 hr as the suspended solids gradually dissolved into the biphasic mixture. This mixture was transferred to a separating funnel and diluted with EtOAc (100 mL) and brine (10 mL), and the aqueous layer was extracted using 10% IpOH/EtOAc (4×50 mL). The organic layers were pooled and washed with brine (10 mL), dried over Na₂SO₄, and evaporated to yield a faintly blue crude solid. This crude solid was dissolved in a mixture of methanol (0.5 mL) and dichloromethane (6 mL) and purified on a Biotage® SNAP Ultra 100 g silica gel column (2-20% MeOH in CH₂Cl₂ over 10 column volumes, followed by an 8-column volume plateau at 20% MeOH). The product eluted as a broad peak after 1-2 column volumes at ˜20% MeOH in CH₂Cl₂. Fractions containing the desired material were pooled and concentrated under reduced pressure to give the title compound as a white solid (1.105 g, 83%). MS m/z obs.=555.9 ((M+2)/2), 1109.8 (M+1).

### 7.11 (S)-2-((S)-2-amino-3-methylbutanamido)-N-(4-(N-((2R,3R)-3-((S)-1-((3R,4S,5R)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-2-methylpropanoyl)sulfamoyl)phenyl)-5-ureidopentanamide (Compound 13)

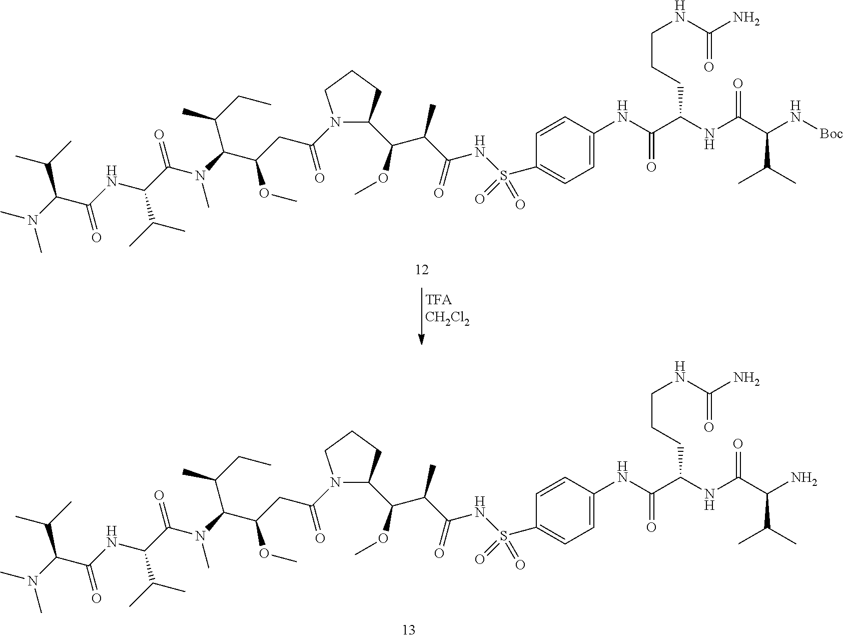 [SMILES: CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](NC(=O)OC(C)(C)C)C(C)C)cc1)OC)N(C)C(=O)[C@@H](NC(=O)[C@H](C(C)C)N(C)C)C(C)C ; CC[C@H](C)[C@@H]([C@@H](CC(=O)N1CCC[C@H]1[C@H](OC)[C@@H](C)C(=O)NS(=O)(=O)c1ccc(NC(=O)[C@H](CCCNC(N)=O)NC(=O)[C@@H](N)C(C)C)cc1)OC)N(C)C(=O)[C@@H](NC(=O)[C@H](C(C)C)N(C)C)C(C)C]

To a solution of Compound 12 (0.926 g, 0.834 mmol) was added a mixture of dichloromethane (10 mL) and trifluoroacetic acid (2.0 mL). The reaction was monitored by HPLC-MS for consumption of starting material (˜45 minutes). The reaction was co-evaporated with acetonitrile (2×10 mL) and dichloromethane (2×10 mL) under reduced pressure to remove excess trifluoroacetic acid. The resulting residue was dissolved in a minimal amount of dichloromethane and methanol (3:1, v/v, −2 mL), and added to a stirred solution of diethyl ether (200 mL) and hexanes (100 mL) dropwise via pipette, producing a suspension of light white solids. The solids were filtered and dried under vacuum to afford the title compound in the form of a white powder, as the trifluoroacetate salt (1.04 g, quantitative yield with some residual solvents). MS m/z obs.=505.8 ((M+2)/2).

### 7.12 (S)—N-(4-(N-((2R,3R)-3-((S)-1-((3R,4S,5R)-4-((S)-2-((S)-2-(dimethylamino)-3-methylbutanamido)-N,3-dimethylbutanamido)-3-methoxy-5-methylheptanoyl)pyrrolidin-2-yl)-3-methoxy-2-methylpropanoyl)sulfamoyl)phenyl)-2-((S)-1-(2,5-dioxo-2,5-dihydro-1H-pyrrol-1-yl)-14-isopropyl-12-oxo-3,6,9-trioxa-13-azapentadecanamido)-5-ureidopentanamide (Linker-Toxin A)

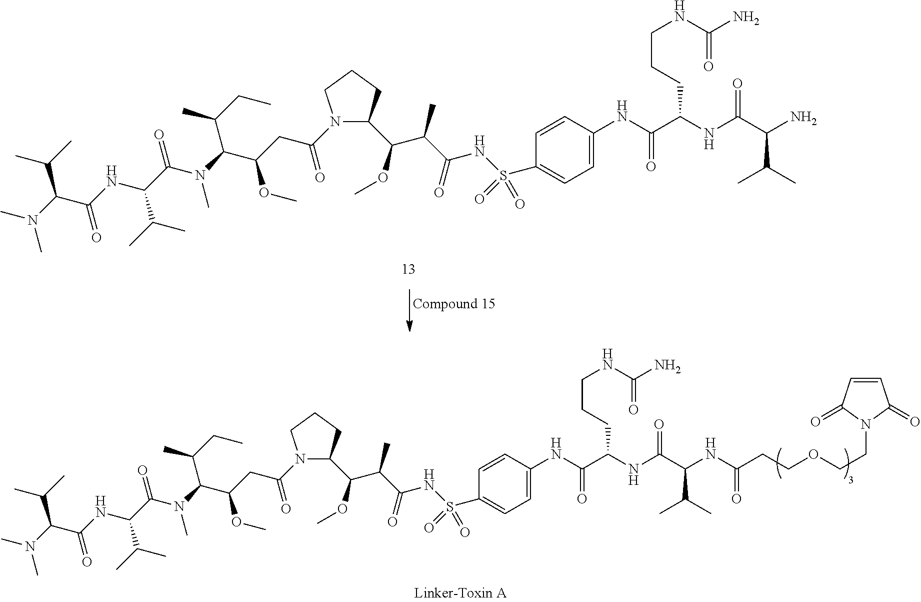

To a stirred solution of Compound 13 (0.722 g, 0.584 mmol) in N,N-dimethylformamide (4 mL) was added Compound 15 (0.314 g, 1.2 equiv) and diisopropylethylamine (0.305 mL, 3.0 equiv). HPLC-MS analysis at 2 h indicated no remaining starting material. The reaction was acidified with TFA (300 μL) and then diluted with diH₂O+0.1% TFA (9 mL). The resultant solution was loaded onto a 120 g Biotage® SNAP Ultra C18 column (Biotage, Uppsala, Sweden) and purified under an ACN/H₂O+0.1% TFA gradient: 20-60% ACN over 10 column volumes, 60-100% ACN over 5 column volumes. Product eluted near 40% ACN. Pure fractions as identified by LCMS were pooled and lyophilized. A white powder solid was recovered from the lyophilizer. The lyophilization was repeated at higher concentration (approx. 50 mg/mL in 2:1 H₂O/ACN) into a vial to produce the title compound as a denser, less flocculant lyophilized solid (754.2 mg, 91%). MS m/z obs.=647.4 ((M+2)/2), 1292.8 (M+1).

### Example 2: ADC Synthesis

Antibody-Drug Conjugates (ADCs) of anti-TF antibodies and Linker-Toxin A, as prepared in Example 1, were prepared as described below.

Briefly, 5 to 10 mg/mL of 25A3 antibody (see Tables 1 and 2 for CDR and V region sequences of clone 25A3) in phosphate-buffered saline (PBS), pH 7.4 was reduced by the addition of Tris(2-carboxyethyl)phosphine (2.0-2.5 or 3.2 molar equivalents) and a final concentration of 0.8 mM diethylenetriamine-pentaacetic acid. After 2 hr at 37° C., the partially reduced antibody was cooled on ice for 10 minutes, then conjugated for 1 h with 8 molar equivalents of Linker-Toxin A on ice. The reaction was quenched with an excess of N-acetyl-L-cysteine. The quenched reaction was allowed to sit on ice for 30 minutes prior to purification. ADCs were purified through two rounds of 40 kDa MWCO Zeba™ Spin Desalting Columns (10 mL Columns, Product #8772, Lot #RL240689) each, as per the manufacturer's protocol. Prior to purification, both sets of columns were primed with sterile PBS. The ADC was purified through one set of PBS primed columns first, the sample was then collected and purified a second time through the other set. After the second purification, the ADC was pooled back together and sterile filtered and frozen at −80° C.

Drug-antibody ratio (DAR) may be measured by UV/vis spectroscopy, hydrophobic interaction chromatography (HIC), and/or reverse phase liquid chromatography separation with time-of-flight detection and mass characterization {RP-UPLC/Mass spectrometry), as described in WO 2016/041082. Distribution of drug-linked forms (for example, the fraction of DAR0, DAR1, DAR2, etc. species) may also be analyzed by various techniques known in the art, including MS (with or without an accompanying chromatographic separation step), hydrophobic interaction chromatography, reverse-phase HPLC or iso-electric focusing gel electrophoresis (IEF), as also described in WO 2016/041082.

For this example, the drug-antibody ratio (DAR) of the resulting ADCs was ˜3. The DAR was determined by hydrophobic interaction chromatography: Average DAR=(0×(DAR0 Area %)+2×(DAR2 Area %)+4×(DAR4 Area %)+6×(DAR6 Area %)+8×(DAR8 Area %)/100. Size exclusion chromatography was used to ensure the ADC preparation was at least 95% monomeric.

ADCs comprising 25A3 and LT-A (25A3-LT-A), e.g. as prepared in this example, were used in the studies of Example 3 below.

### Example 3: Development of Formulation

The purpose of this formulation development study was to examine the effects of buffer type, concentration, and pH on the stability of the solution formulation. The goal was to develop a stable, frozen solution of 25A3-LT-A prepared in Example 2.

### Preparation of Solution Formulations

A total of 15 different solution formulations were tested in the study. The buffer preparations were shown in Table 3, in which three different buffers—histidine, phosphate, and citrate were prepared at three concentrations—10 mM, 30 mM and 50 mM at three pH levels—pH 5, pH 6, and pH 7. The formulations 1-15 in Table 3 were prepared from the 25A3-LT-A 4.6 mg/mL aqueous buffer solution. The 25A3-LT-A solution was concentrated and dialyzed in the designed buffer. A final formulation was adjusted to a concentration as close to 20 mg/mL as possible. The 25A3-LT-A formulations were filtered using a 0.2 μm syringe disc filter.

TABLE 3
Formulations Used in the Study

| | | Concentration | |
|---|---|---|---|
| Formulation | Buffer Type | [mM] | pH |
| 1 | Histidine | 50 | 7 |
| 2 | Histidine | 10 | 5 |
| 3 | Phosphate | 10 | 7 |
| 4 | Citrate | 10 | 6 |
| 5 | Citrate | 10 | 5 |
| 6 | Citrate | 50 | 5 |
| 7 | Phosphate | 50 | 6 |
| 8 | Phosphate | 10 | 6 |
| 9 | Phosphate | 50 | 7 |
| 10 | Citrate | 50 | 6 |
| 11 | Histidine | 10 | 7 |
| 12 | Histidine | 50 | 5 |
| 13 | Histidine | 30 | 6 |
| 14 | Citrate | 30 | 6 |
| 15 | Phosphate | 30 | 6 |

### Analysis of Formulation Stability

The stability of each formulation was tested in individual vial containing 1 mL of solution. The vials were placed at temperatures ranging from −80° C. to 40° C. for up to six months. The analysis of UV/Vis, size-exclusion chromatography (SEC), Dynamic Light Scattering (DLS), and imaged capillary electrophoresis (iCE) was performed at TO and each of the time points as described in Table 4.

TABLE 4
Storage Conditions and Testing Time Points for the Stability Study

| Storage Condition | Time Points |
|---|---|
| −80° C./Ambient RH | 2 months, 4 months, and 6 months |
| −20° C./Ambient RH | 1 month, 2 months, 4 months, and 6 months |
| 2-8° C./Ambient RH | 2 weeks, 1 month, and 2 months |
| 25° C./60% RH | 1 week and 2 weeks |
| 40° C./75% RH | 1 week and 2 weeks |

### Test 1: Concentration Measurement by UV/Vis

The protein concentration was measured in UV/Vis at 280 nm for the drug product formulations, with a control of 125 μL reference sample (L/N E01-10). The concentration of each formulation was tested at TO and after storage at the conditions shown in Table 4. The results in Table 5 showed that the concentrations of formulations remained near the expected value of 20 mg/mL for each formulation throughout the study.

TABLE 5
Concentrations for Formulations Throughout the Stability Study Tested by UV/Vis

| | | Concentration (mg/mL) | | | | | | | | | | | | | |
|---|---|---|---|---|---|---|---|---|---|---|---|---|---|---|---|
| | | 40° C. | | 25° C. | | 2° C.-8° C. | | | −20° C. | | | | −80° C. | | |
| | | 6 | 13 | 6 | 13 | 13 | 1 | 2 | 1 | 2 | 4 | 6 | 2 | 4 | 6 |
| Formulation | T0 | days | days | days | days | days | mon | mos | mon | mos | mos | mos | mos | mos | mos |
| 1 | 19.4 | 19.5 | 19.8 | 19.4 | 19.4 | 19.4 | 19.6 | 19.4 | 19.6 | 19.4 | 19.3 | 19.4 | 19.3 | 19.5 | 19.4 |
| 2 | 19.8 | 19.7 | 19.8 | 19.7 | 19.8 | 19.7 | 19.9 | 19.8 | 19.9 | 19.8 | 19.8 | 20 | 19.7 | 19.7 | 19.7 |
| 3 | 19.6 | 19.6 | 19.7 | 19.6 | 19.6 | 19.6 | 19.8 | 19.6 | 19.7 | 19.5 | 19.5 | 19.6 | 19.6 | 19.5 | 19.7 |
| 4 | 19.6 | 19.6 | 19.8 | 19.6 | 19.7 | 19.7 | 19.9 | 19.7 | 19.8 | 19.5 | 19.6 | 19.6 | 19.7 | 19.6 | 19.6 |
| 5 | 19.7 | 19.7 | 19.8 | 19.8 | 19.7 | 19.7 | 19.9 | 19.7 | 20 | 19.7 | 19.8 | 19.7 | 19.8 | 20.1 | 19.8 |
| 6 | 19.8 | 19.8 | 19.9 | 19.8 | 19.9 | 19.8 | 20 | 19.8 | 20 | 19.7 | 19.8 | 19.8 | 19.8 | 19.8 | 19.8 |
| 7 | 19.8 | 19.8 | 19.9 | 19.8 | 19.8 | 19.8 | 20 | 19.8 | 19.9 | 19.8 | 19.8 | 19.8 | 19.9 | 19.8 | 19.9 |
| 8 | 19.8 | 19.8 | 20 | 19.8 | 19.8 | 19.8 | 20.1 | 19.7 | 19.9 | 19.7 | 19.7 | 19.8 | 19.8 | 19.8 | 19.8 |
| 9 | 19.8 | 19.9 | 19.9 | 19.9 | 20 | 19.9 | 20.2 | 19.9 | 20.1 | 19.9 | 19.8 | 19.9 | 19.9 | 19.7 | 19.7 |
| 10 | 19.7 | 19.7 | 19.7 | 19.7 | 19.7 | 19.7 | 20 | 19.7 | 19.9 | 19.5 | 19.7 | 19.7 | 19.7 | 19.7 | 19.7 |
| 11 | 19.5 | 19.6 | 19.6 | 19.5 | 19.6 | 19.5 | 19.8 | 19.6 | 19.8 | 19.6 | 19.6 | 19.6 | 19.6 | 19.6 | 19.5 |
| 12 | 19.8 | 19.7 | 19.8 | 19.8 | 19.8 | 19.8 | 20.1 | 19.7 | 20 | 19.7 | 19.9 | 19.7 | 19.8 | 19.8 | 19.8 |
| 13 | 19.7 | 19.7 | 19.7 | 19.6 | 19.8 | 19.7 | 19.9 | 19.6 | 19.9 | 19.6 | 19.7 | 19.6 | 19.6 | 19.6 | 19.7 |
| 14 | 19.5 | 19.5 | 19.6 | 19.5 | 19.6 | 19.5 | 19.7 | 19.5 | 19.8 | 19.6 | 19.5 | 19.5 | 19.5 | 19.5 | 19.5 |
| 15 | 19.6 | 19.7 | 19.8 | 19.7 | 19.8 | 19.7 | 20 | 19.7 | 19.9 | 19.7 | 19.7 | 19.8 | 19.7 | 19.7 | 19.7 |

### Test 2: Size Heterogeneity Analysis by SEC

The molecular size distribution of 25A3-LT-A was assessed by SEC. The mobile phase was prepared using sodium chloride, sodium phosphate, dibasic and sodium phosphate monobasic and adjusted to pH 6.8. A blank was prepared by combining 77 μL of the 30 mM histidine buffer prepared at pH 6.0 with 123 μL of mobile phase to match the dilution used for the reference sample. Samples were combined with mobile phase and treated in a similar manner as the blank.

The formulations exhibited different degrees in decreases in the percent monomer and increases in the percent high molecular weight (HMWV) species under different storage conditions. For example, formulation 9 that was prepared in a 50 mM phosphate buffer at pH 7 exhibited a 0.6% decrease in monomer and a 0.7% increase in HMW after storage for 6 months at −20° C. In contrast, this formulation exhibited a 2.4% decrease in monomer and a 2.5% increase in HMW after storage for 6 months at −80° C.

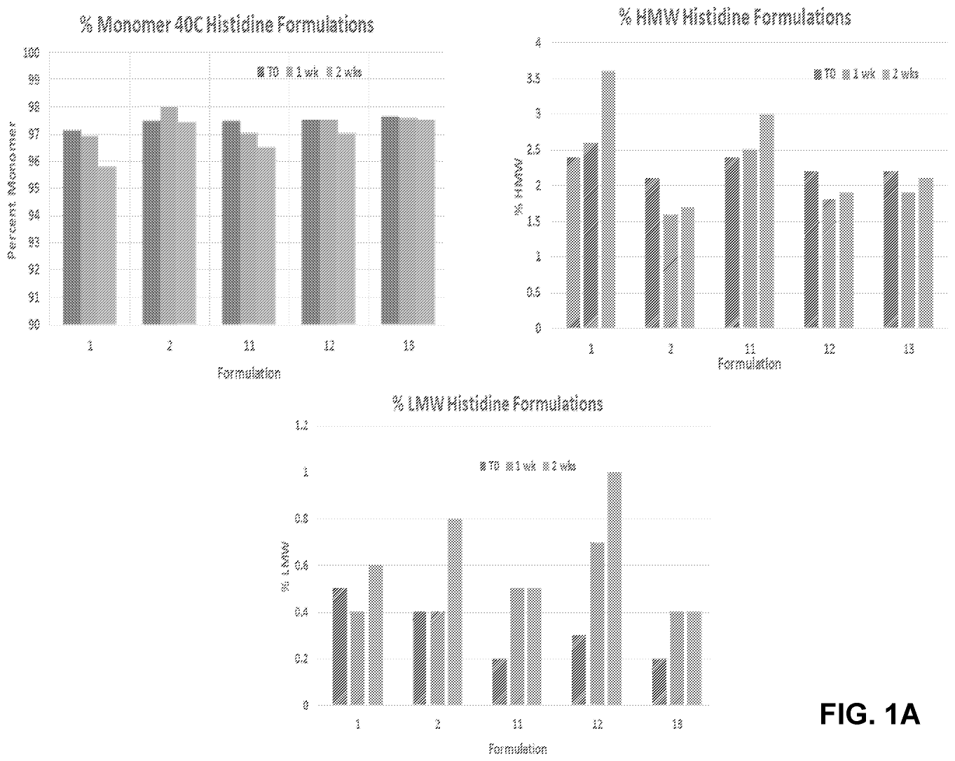

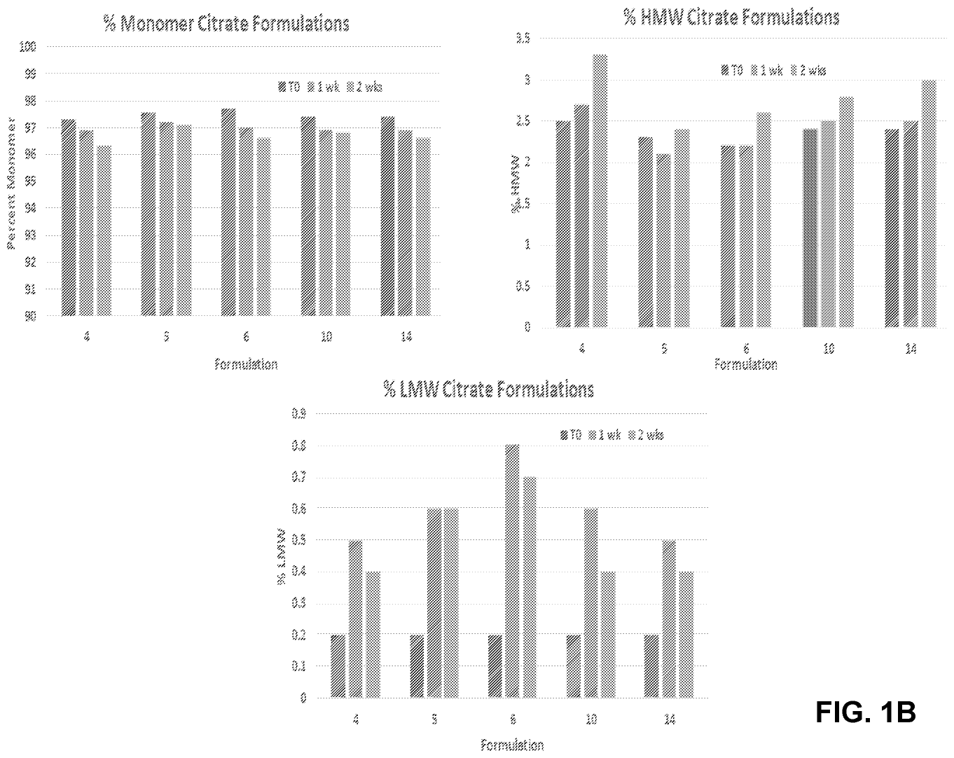

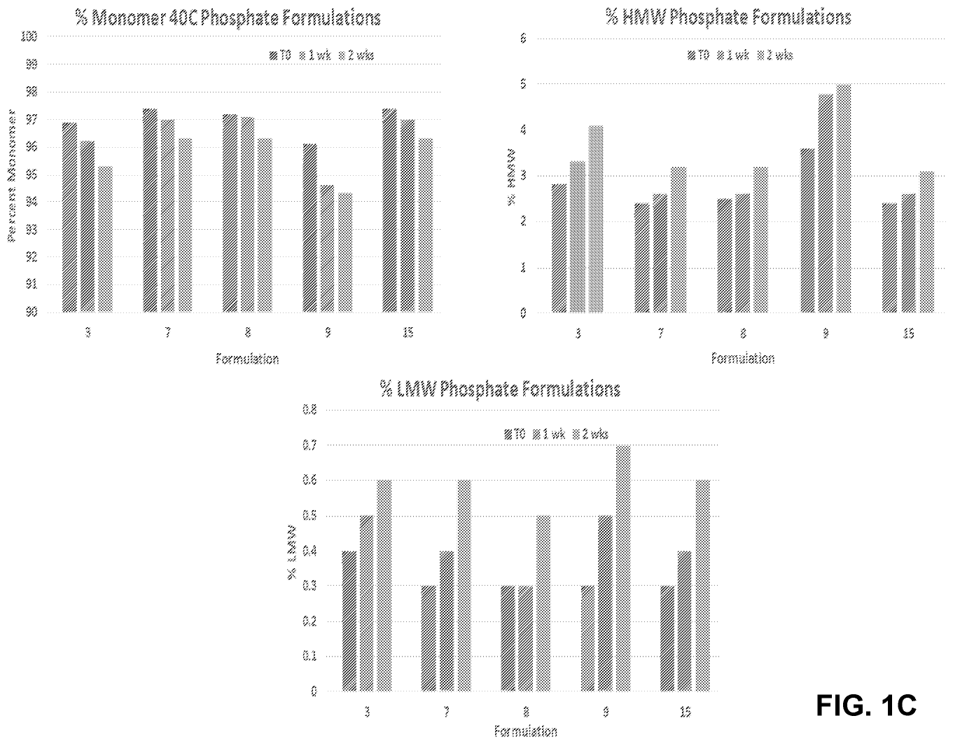

The formulations prepared in histidine stored at 2° C. to 8° C. for up to 2 months exhibited no significant changes in percent monomer, percent HMW, or percent low molecular weight (LMW) species during storage. No significant changes were observed in storage at 25° C./60% RH for up to 2 weeks too. However, changes in the SEC data were apparent for the formulations stored at 40° C./75% RH for up to 2 weeks, except for the histidine formulations. FIGS. 1A-1C show that the formulations prepared with histidine (FIG. 1A) had fewer changes in percentage of species than for the formulations prepared with citrate (FIG. 1B) and phosphate (FIG. 1C).

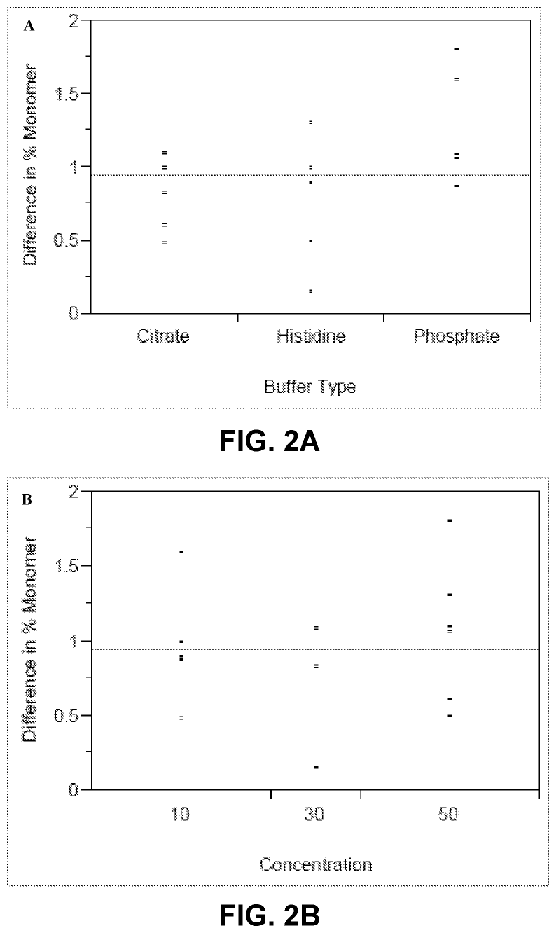

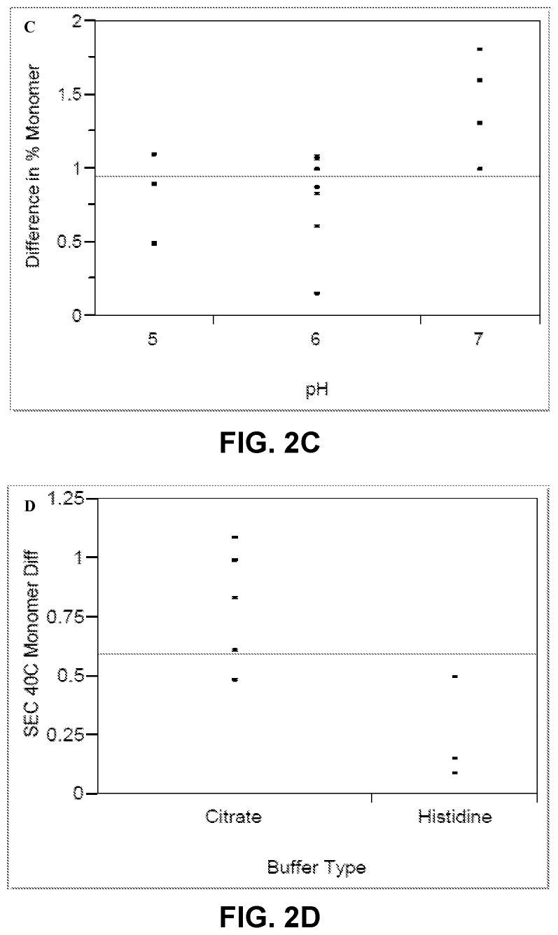

The significance of the difference in percent monomer between TO and after storage at 40° C. for 2 weeks was further examined statistically by a one-way analysis of variance. The analysis was conducted based on the buffer type, buffer concentration, and formulation pH, respectively. The results indicate that: (1) the formulations prepared with citrate and histidine had fewer changes in the percent monomer than the formulation prepared with phosphate (FIG. 2A); (2) 30 mM concentration exhibited fewer changes in percent monomer than the 10 mM and 50 mM concentrations (FIG. 2B); and (3) smaller differences in percent monomer occurred at pH 5 and pH 6 (FIG. 2C).

The data suggested that the ADC provided herein was less stable in a phosphate buffer, therefore, the statistical analysis was re-examined only in the formulations prepared with citrate and histidine. FIG. 2D showed that the formulations prepared with histidine exhibited less change in percent monomer during storage at 40° C. for up to 2 weeks. The statistics showed samples stored at 40° C. exhibited no significant difference in percent monomer on the basis of buffer concertation or pH.

In summary, based on the SEC data of the percent monomer changes for formulations stored at 40° C./75% RH for up to 2 weeks, higher molecule stability was observed when the formulations were prepared using a histidine buffer, at pH less than 7, and with a buffer concentration between approximately 10 mM and 50 mM. Also, formulations 3, 9, and 12 exhibited changes in percent monomer during storage at −20° C. and −80° C. for up to 6 months. The observation was consistent because formulations 3 and 9 were prepared with a less stable phosphate buffer. The changes in formulation 12 that was prepared with a 50 mM histidine buffer at pH 5 may suggest a further adjustment in histidine concentration.

### Test 3: Charge Heterogeneity Analyzed by iCE

The charge heterogeneity of 25A3-LT-A was assessed using iCE. The iCE diluent was prepared by combining Pharmalyte 3-10, Pharmalyte 8-10, Pharmalyte 5-8, 1% methyl cellulose, pI marker 5.85, pI marker 9.50, and 10 M urea in a Falcon tube. Samples were prepared in duplicate by adding 10 μL of the sample to 90 μL of the diluent. All iCE comparisons were presented with line graphs. The percent main pI peak shift indicated the charge heterogeneity changes of 25A3-LT-A.

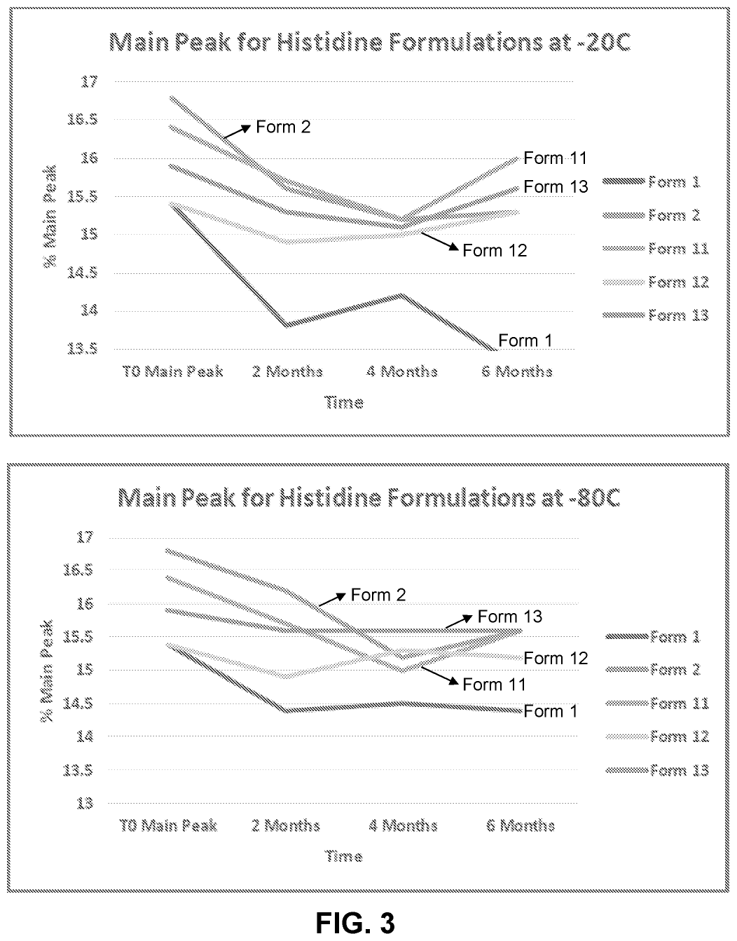

Among the formulations prepared with histidine, no large changes in percent main peak, percent acidic species, or percent basic species were observed during storage for up to 6 months at −20° C. and −80° C. However, formulation 1 that was prepared with 50 mM histidine at pH 7 exhibited a consistent decrease in percent main peak (FIG. 3). Formulation 1 also exhibited a decrease in percent main peak and acidic species during storage at 2° C. to 8° C. for up to 2 months. Therefore, the results suggested that a formulation with 50 mM histidine at pH 7 may be less stable. This was consistent with the SEC analysis which also suggested a less stability in formulation 12 that was prepared with a 50 mM histidine buffer at pH 5.

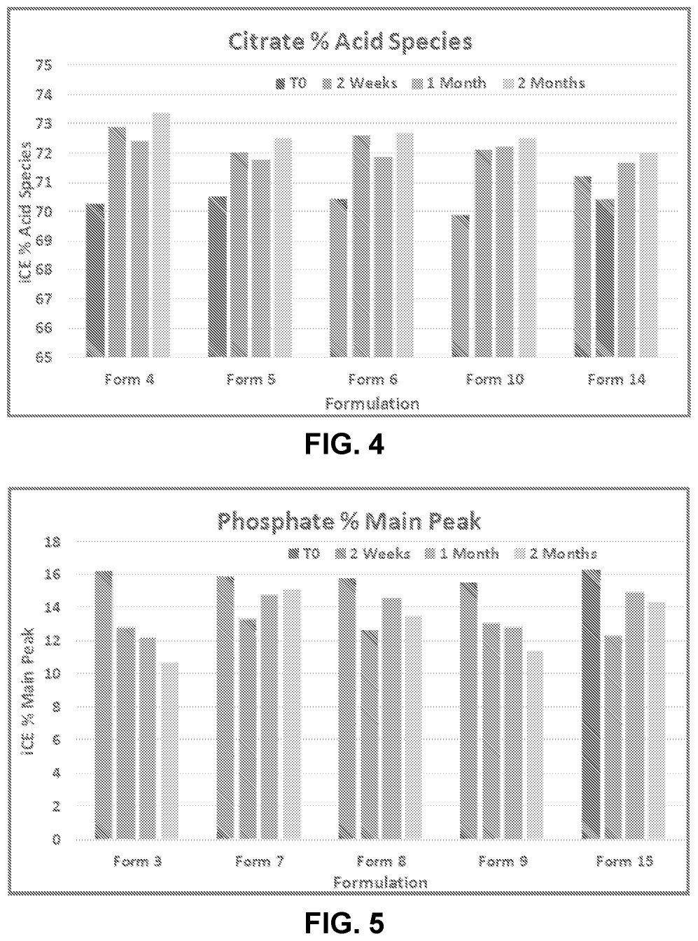

The formulations prepared with citrate or phosphate during storage at 2° C. to 8° C. for up to 2 months showed changes in percent main peak, percent acidic species, or percent basic species. As shown in FIG. 4, the percent acidic species increased in all of the formulations prepared with citrate during storage at 2° C. to 8° C. for up to 2 months. The citrate formulations 4 and 10 exhibited decreases in percent main peak of approximately 2%. In all formulations prepared with phosphate buffer, increases in the acidic species were also observed. FIG. 5 showed that all formulations prepared with the phosphate during storage at 2° C. to 8° C. for up to 2 months exhibited decreases in the percent main peak, especially formulations 3 and 9. Formulations 3 and 9 were prepared at pH 7 with buffer concentrations of 10 mM and 50 mM, respectively. These results again suggested that pH 7 may not be a condition that facilitates the formulation stability.

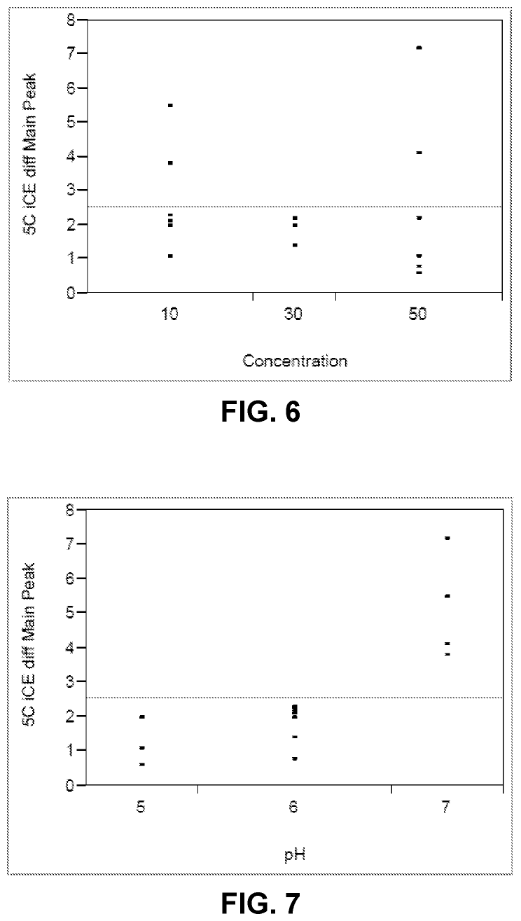

The one-way analysis of variance was applied to examine changes in percent main peak and percent acidic species for formulations stored at 2° C. to 8° C. for up to 2 months. The analysis suggested that the formulations prepared with citrate buffer showed less change in percent main peak and percent acidic species. The data also suggested that fewer changes in percent main peak were observed for formulations stored at 2° C. to 8° C. for up to 2 months when the buffer concentration was 30 mM (FIG. 6). In addition, the formulations prepared at pH 5 to pH 6 in the same storage condition showed fewer changes in percent main peak (FIG. 7).

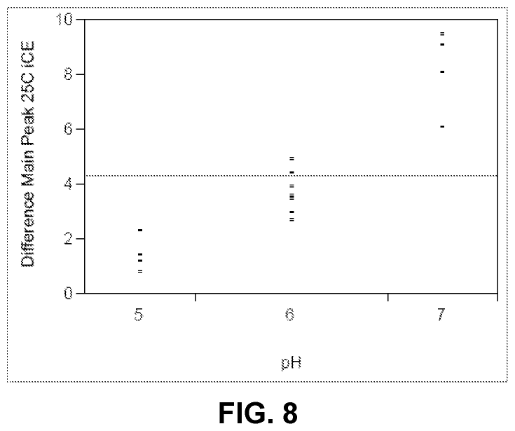

In the condition of 25° C. storage for up to 2 weeks, all formulations prepared with citrate or phosphate buffer exhibited increases in percent acidic species. Formulations 1 and 11 that were prepared at pH 7 showed large decreases in percent main peak. In the one-way analysis of variance, the data suggested that no differences in percent main peak were observed between phosphate and citrate buffer on the basis of buffer type or buffer concentration. The data show fewer changes expected for formulations prepared in the citrate buffer at 30 mM with a pH between 5 and 6. Also, among formulations prepared with citrate or phosphate buffer, the analysis showed that the formulations prepared at pH 5 exhibited fewer changes (FIG. 8). These results suggested that a buffer concentration of 30 mM at pH 5 to 6 may be a better condition to stabilize the formulations.

During the storage at 40° C. for up to 2 weeks, all formulations prepared with the phosphate buffer exhibited large decreases in percent main peak. All formulations exhibited increases in iCE percent acidic peaks and decreases in iCE percent basic peaks. In contrast, formulation 12 prepared with histidine and formulation 6 prepared with citrate exhibited fewer changes in percent main peak during storage at 40° C. for up to 2 weeks. Both formulations were prepared with 50 mM buffer concentration at pH 5. The results suggested that the 50 mM histidine or citrate buffer at pH 5 may be a better condition to stabilize the formulations.

The one-way analysis of variance was applied to examine the difference in percent main peak between TO and after storage at 40° C. for up to 2 weeks. The data suggested that fewer changes in iCE percent main peak were observed for formulations prepared in citrate or histidine buffers at pH 5 and 6. No large differences in the analysis were observed on the basis of buffer concentration. Also, formulations prepared in citrate or histidine buffers at pH 5 or 6 exhibited the least changes after storage at 40° C. for up to 2 weeks.

In summary, the iCE charge heterogeneity data suggested that formulations prepared in histidine buffers at pH 5 or 6 exhibited the least changes. Buffer concentration did not significantly influence the analysis, however, the data suggested that a 30 mM buffer concentration may be an appropriate concentration for a midpoint for the formulation.

### Test 4: Particle Size Analyzed by DLS

DLS was used to evaluate changes in the average particle size and percent polydispersity of each formulation at TO and after storage. Each sample vial was gently agitated by swirling for approximately 10 seconds and loaded into sample plate. The samples were analyzed in triplicate with 10 acquisitions for each analysis. Changes were considered large if the average particles size and/or polydispersity double or triple over the storage time. They may also be considered large if the particle size distribution became multi-modal.

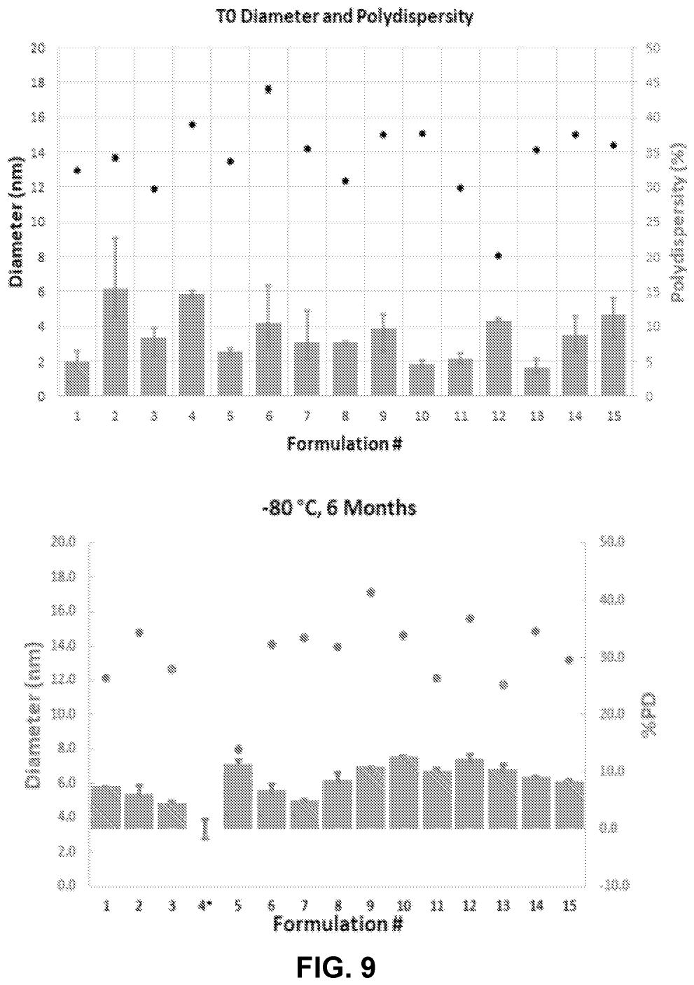

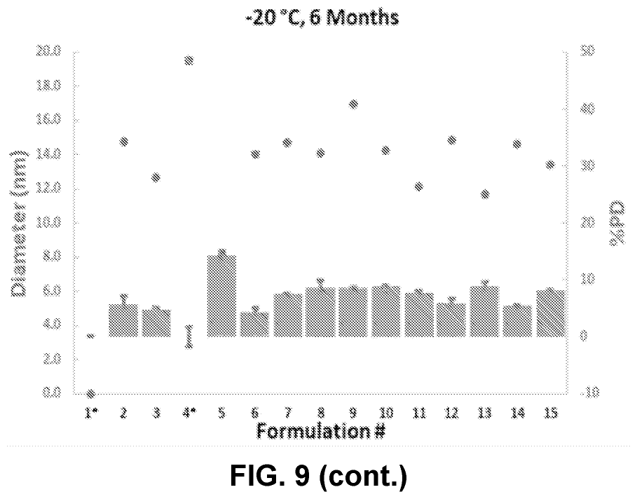

No large changes in the average particle size or percent polydispersity were observed for the tested formulations, except for formulations 1 and 4 (FIG. 9). The particle size distribution for formulation 4 became multimodal after storage at −80° C. and −20° C. for 6 months. Formulation 1 became multi-modal after 4 and 6 months of storage at −20° C. Formulation 4 was prepared in 10 mM citrate at pH 6. Formulation 1 was prepared in 50 mM histidine at pH 7. The results were consistent with the observation that formulation 1 was less stable in the iCE analysis. A large increase in the percent polydispersity for formulation 8 was observed after storage at 40° C. for up to 2 weeks. Formulation 8 was prepared in 10 mM phosphate at pH 6. No large changes in average particle size or percent polydispersity were observed for the other formulations after storage at 40° C. for up to 2 weeks.

In conclusion, the data suggested that histidine buffer offered the best stability for the molecule within a pH range of 5 to 6. The buffer concentration appeared acceptable within a range of 10 mM to 50 mM.

From the formulation studies described above based on the preparation and analysis of numerous formulations with variations of pH (5, 6 and 7), excipient (phosphate, citrate and histidine) and concentration (10 mM, 30 mM, 50 mM), a final formulation (Table 6) was selected of 20 mM Histidine at pH 5.5, along with additions of sucrose and polysorbate 80, which demonstrated surprisingly superior stability. Histidine acts as a buffering agent and hydrochloric acid is used for pH control. Sucrose acts as a tonicifier and stabilizes the ADC in solution during storage. Polysorbate 80 is a surfactant and also stabilizes injection during manufacturing and storage.

TABLE 6
Composition of 25A3-LT-A Final Dosage Form

| Component¹ | Concentration | Function | Quality Standard |
|---|---|---|---|
| 25A3-LT-A | 10 mg/mL | Active drug | Internal |
| | | substance | specification |
| L-Histidine | 20 mM | Buffer | USP, Ph. Eur. |
| HCl 1N¹ | about 0.55 mg/mL | pH control | Ph.Eur. |
| Sucrose | 8% w/v | Tonicifier | NF, Ph. Eur. |
| Polysorbate 80 | 0.2% w/v | Surfactant | NF, Ph. Eur. |
| Water for | q.s. | Diluent | USP, Ph. Eur. |
| Injection | | | |

USP = United States Pharmacopeia; NF = National Formulary
¹HCl 1N is used to titrate the histidine buffer to the target pH so amounts may vary from batch to batch.

### Example 4: Stability of the 25A3-LT-A Dosage Form

A 25A4-LT-A composition was prepared as described in Table 6. The stability of the prepared composition was tested under a variety of storage conditions, such as (i)<−65° C. (a first long-term storage condition), (ii) −20° C.±5° C. (a second long-term storage condition), (iii) 25±2° C./60±5% relative humidity (RH) (a first accelerated storage condition), (iv) 5±3° C. (a second accelerated storage condition), (v) about 25° C./60% RH (a third accelerated storage condition), and (vi) 40° C./75% RH (a severe storage condition). Various storage time periods were investigated, such as 0 day (initial), 1 day, 1 week, 2 weeks, 1 month, 3 months, 6 months, 9 months, 12 months, 18 months, and 23 months, while additional storage time periods of 24 months, 30 months, 36 months, 48 months and 60 months are under investigation.

No significant changes in pH, protein concentration (tested by UV), antigen binding (tested by Enzyme-Linked Immunosorbent Assay, ELISA), purity (tested by High Performance Liquid Chromatography, HPLC or Capillary Electrophoresis Sodium Dodecyl Sulfate, CE-SDS), charge variants (tested by Imaged Capillary Isoelectric Focusing, icIEF), aggregation or size variants (tested by Size Exclusion Chromatography, SEC), or other attributes of the composition were observed up to 23 months of storage at the storage condition of <−65° C. Data also showed no change over 23 months at the proposed long-term storage temperature of −20° C. In addition, the data generated at the intended storage condition showed no significant trend over time for any tested attribute. For example, the DAR of the composition remained at about 3.8.

A number of embodiments have been described herein. It should be understood, however, that various modifications may be made without departing from the spirit and scope of the present invention. Accordingly, other embodiments are within the scope of the following claims.

What is claimed is:

1. A pharmaceutical composition comprising
(i) an antibody-drug conjugate of the following Formula (I): 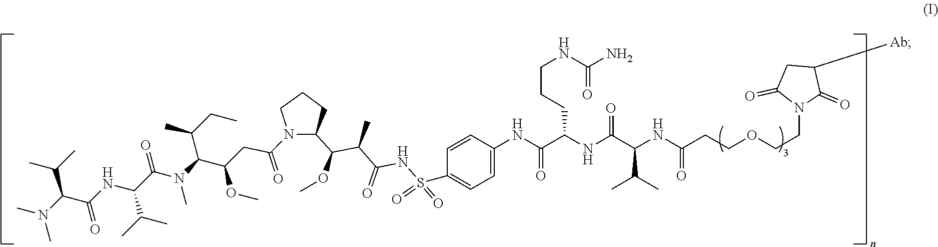
or a pharmaceutically acceptable salt thereof,
wherein:
Ab is a tissue factor (TF) antibody, wherein the Ab comprises a VH-CDR1, a VH-CDR2, a VH-CDR3, a VL-CDR1, a VL-CDR2, and a VL-CDR3 of the VH-CDR1, VH-CDR2, VH-CDR3, VL-CDR1, VL-CDR2, and VL-CDR3 of the antibody designated 25A3;
n is an integer greater than or equal to 1; and
the succinimidyl group is attached to the Ab through a covalent bond;
(ii) a buffer;
(iii) a tonicifier; and
(iv) a surfactant.

2. The pharmaceutical composition of claim 1, wherein the succinimidyl group is attached to the Ab via the cysteine residues of the Ab.

3. The pharmaceutical composition of claim 1 or claim 2, wherein n is selected from the group consisting of 1, 2, 3, and 4, optionally wherein n is selected form the group consisting of 3 and 4.

4. The pharmaceutical composition of any one of claims 1 to 3, wherein the drug-antibody-ratio (DAR) of the antibody-drug conjugate is about 1 to about 4, optionally about 2 to about 4, further optionally about 3, yet further optionally about 3.8.

5. The pharmaceutical composition of any one of claims 1 to 4, wherein the Ab comprises a VH comprising the amino acid sequence of SEQ ID NO:37 and a VL comprising the amino acid sequence of SEQ ID NO:38.

6. The pharmaceutical composition of any one of claims 1 to 5, wherein the Ab comprises:
a heavy chain comprising the amino acid sequence
of
(SEQ ID NO: 40)
QVQLVQSGAEVKKPGASVKVSCKASGYTFDVYGISWVRQAPGQGLEW
MGWIAPYSGNTNYAQKLQGRVTMTTDTSTSTAYMELRSLRSDDTAVYY
CARDAGTYSPFGYGMDVWGQGTTVTVSSASTKGPSVFPLAPSSKSTS
GGTAALGCLVKDYFPEPVTVSWNSGALTSGVHTFPAVLQSSGLYSLSSV
VTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVM
HEALHNHYTQKSLSLSPG;
and
a light chain comprising the amino acid sequence
of
(SEQ ID NO: 41)
DIQMTQSPSTLSASVGDRVTITCQASQSINNWLAWYQQKPGKAPKLLIY
KAYNLESGVPSRFSGSGSGTEFTLTISSLQPDDFATYYCQLFQSLPPFT
FGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPVTKSFNRGEC

7. The pharmaceutical composition of any one of claims 1 to 6, wherein the buffer is histidine, the tonicifier is sucrose, and the surfactant is polysorbate 80; and wherein the pharmaceutical composition further comprises hydrochloric acid.

8. The pharmaceutical composition of any one of claims 1 to 7, comprising 5-20 mg/mL of the antibody-drug conjugate, 10-50 mM histidine, 5-10% (w/v) sucrose, and 0.01-0.05% (w/v) polysorbate 80, and wherein the pharmaceutical composition has a pH value of between 5 to 6.

9. The pharmaceutical composition of any one of claims 1 to 8, comprising about 10 mg/mL of the antibody-drug conjugate, about 20 mM histidine, about 8% (w/v) sucrose, and about 0.02% (w/v) polysorbate 80, and wherein the pharmaceutical composition has a pH value of about 5.5.

10. The pharmaceutical composition of any one of claims 1 to 9 for use in treating a disease or disorder in a subject.

11. A method of treating a disease or disorder in a subject comprising administering the pharmaceutical composition of any one of claims 1 to 9 to the subject.

12. The pharmaceutical composition of claim 10 or the method of claim 11, wherein the disease or disorder is cancer, optionally selected from the group consisting of non-small cell lung cancer (NSCLC), urothelial cancer, ovarian cancer (e.g., epithelial), cervical cancer (e.g., with squamous cell or adenocarcinoma histology), head and neck cancer (e.g., with squamous cell histology), and pancreatic cancer.

13. The pharmaceutical composition or the method of any one of claims 10 to 12, wherein the subject is a human subject.

14. The pharmaceutical composition of any one of claims 1 to 9, wherein the pharmaceutical composition is lyophilized.

15. The pharmaceutical composition of any one of claims 1 to 9 and 14, wherein the pharmaceutical composition is stored in a glass vial or a polycarbonate bottle.
